(19) United States
(12) Patent Application Publication
(10) Pub. No.: US 2022/0395496 A1
(43) Pub. Date: Dec. 15, 2022

(54) PIPERIDINYL AMINE COMPOUNDS FOR THE TREATMENT OF AUTOIMMUNE DISEASE

(71) Applicant: Hoffmann-La Roche Inc., Little Falls, NJ (US)

(72) Inventors: Taishan HU, Shanghai (CN); Buyu KOU, Shanghai (CN); Haixia LIU, Shanghai (CN); Hong SHEN, Shanghai (CN); Wei ZHU, Shanghai (CN); Ge ZOU, Shanghai (CN)

(73) Assignee: Hoffmann-La Roche Inc., Little Falls, NJ (US)

(21) Appl. No.: 17/761,150

(22) PCT Filed: Sep. 14, 2020
(86) PCT No.: PCT/EP2020/075585

§ 371 Date: Mar. 16, 2022

(30) Foreign Application Priority Data
Sep. 16, 2019 (CN) PCT/CN2019/105997

(51) Int. Cl. A61K 31/4709; A61K 31/498; A61K 31/4162; A61K 31/428

(52) U.S. Cl. CPC A61K 31/4709; A61K 31/498; A61K 31/4162; A61K 31/428

(57) ABSTRACT

The present invention relates to compounds of formula (I), wherein R¹, R², R³, R⁴ and R⁵ are as described herein, and their pharmaceutically acceptable salt, enantiomer or diastereomer thereof, and compositions including the compounds and methods of using the compounds.

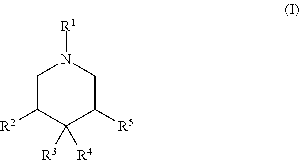

The present invention relates to organic compounds useful for therapy and/or prophylaxis in a mammal, and in particular to antagonist of TLR7 and/or TLR8 and/or TLR9 useful for treating systemic lupus erythematosus or lupus nephritis.

### FIELD OF THE INVENTION

Autoimmune connective tissue disease (CTD) include prototypical autoimmune syndromes such as Systemic Lupus Erythematosus (SLE), primary Sjögren's syndrome (pSjS), mixed connective tissue disease (MCTD), Dermatomyositis/Polymyositis (DM/PM), Rheumatoid Arthritis (RA), and systemic sclerosis (SSc). With the exception of RA, no really effective and safe therapies are available to patients. SLE represents the prototypical CTD with a prevalence of 20-150 per 100,000 and causes broad inflammation and tissue damage in distinct organs, from commonly observed symptoms in the skin and joints to renal, lung, or heart failure. Traditionally, SLE has been treated with nonspecific anti-inflammatory or immunosuppressive drugs. However, long term usage of immunosuppressive drug, e.g. corticosteroids is only partially effective, and is associated with undesirable toxicity and side effects. Belimumab is the only FDA-approved drug for lupus in the last 50 years, despite its modest and delayed efficacy in only a fraction of SLE patients (Navarra, S. V. et al Lancet 2011, 377, 721). Other biologics, such as anti-CD20 mAbs, mAbs against or soluble receptors of specific cytokines, have failed in most clinical studies. Thus, novel therapies are required that provide sustained improvement in a greater proportion of patient groups and are safer for chronic use in many autoimmune as well as auto-inflammation diseases.

Toll Like Receptors (TLR) are an important family of pattern recognition receptors (PRR) which can initiate broad immune responses in a wide variety of immune cells. As natural host defense sensors, endosomal TLRs 7, 8 and 9 recognize nucleic acids derived from viruses, bacteria; specifically, TLR7/8 and TLR9 recognize single-stranded RNA (ssRNA) and single-stranded CpG-DNA, respectively. However, aberrant nucleic acid sensing of TRL7/8/9 is considered as a key node in a broad of autoimmune and auto-inflammatory diseases (Krieg, A. M. et al. Immunol. Rev. 2007, 220, 251. Jiménez-Dalmaroni, M. J. et al Autoimmun Rev. 2016, 15, 1. Chen, J. Q., et al. Clinical Reviews in Allergy & Immunology 2016, 50, 1.) Therefore, TLR7/8/9 represents a new therapeutic target for autoimmune and auto-inflammatory diseases, for which no effective steroid-free and non-cytotoxic oral drugs exist, and inhibition of these pathways from the very upstream may deliver satisfying therapeutic effects. From a safety perspective, because there are multiple nucleic acid sensing pathways (e.g. other TLRs, cGAS/STING), such redundancy should still allow responses to infection in the presence of TLR7/8/9 inhibition. As such, we proposed and invented oral compounds that target and suppress TLR7/8/9 for the treatment of autoimmune and auto-inflammatory diseases.

### SUMMARY OF THE INVENTION

The present invention relates to novel compounds of formula (I) or (Ia),

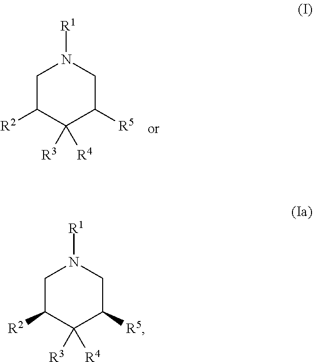

wherein

### R¹ is

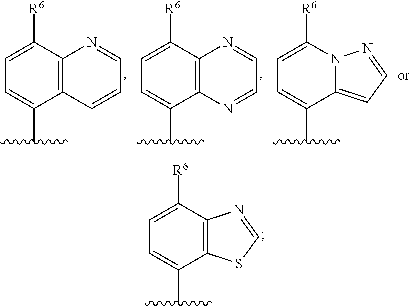

wherein R⁶ is cyano or halogen;
R² is C₁₋₆alkyl;
R³ is H or halogen;
R⁴ is H, halogen or hydroxy;
R⁵ is 1,3-dihydropyrrolo[3,4-c]pyridinyl substituted by piperazinyl;

2,3,3a,7a-tetrahydro-1H-indenylamino substituted by piperazinyl;

3,4-dihydro-1H-isoquinolinyl substituted by piperazinyl;

5,7-dihydropyrrolo[3,4-b]pyridinyl substituted by piperazinyl;

5,7-dihydropyrrolo[3,4-d]pyrimidinyl substituted by piperazinyl;

isoindolinyl substituted by piperazinyl; or

—NR^{5a}R^{5b}; wherein
R^{5a} is H or C₁₋₆alkyl;
R^{5b} is 1,2,3,4-tetrahydroisoquinolinylC₁₋₆ alkyl;
   5,6,7,8-tetrahydro-1,6-naphthyridinylC₁₋₆alkyl;
   isoindolinylC₁₋₆alkyl;
   phenyl(hydroxy)C₁₋₆ alkyl substituted by piperazinyl;
   phenylC₁₋₆alkyl, said phenylC₁₋₆alkyl being substituted by one or two substituents independently selected from (C₁₋₆ alkyl)₂pyridinyl, aminopiperidinyl, aminopyrrolidinyl, C₁₋₆alkylpiperazinyl, halogen, piperazinyl and piperidinyl;
   pyrazinylC₁₋₆ alkyl substituted by piperazinyl;
   pyridazinylC₁₋₆alkyl substituted by piperazinyl;
   pyridinylC₁₋₆ alkyl substituted by piperazinyl or C₁₋₆alkylpiperazinyl; or
   pyrimidinylC₁₋₆ alkyl substituted by piperazinyl or C₁₋₆alkylpiperazinyl;
      or a pharmaceutically acceptable salt, enantiomer or diastereomer thereof.

Another object of the present invention is related to novel compounds of formula (I) or (Ia), their manufacture, medicaments based on a compound in accordance with the invention and their production as well as the use of compounds of formula (I) or (Ia) as TLR7 and/or TLR8 and/or TLR9 antagonist, and for the treatment or prophylaxis of systemic lupus erythematosus or lupus nephritis. The compounds of formula (I) or (Ia) show superior TLR7 and/or TLR8 and/or TLR9 antagonism activity. In addition, the compounds of formula (I) or (Ia) also show good cytotoxicity, solubility, human microsome stability and SDPK profiles, as well as low CYP inhibition.

### DETAILED DESCRIPTION OF THE INVENTION

### Definitions

The term “C₁₋₆ alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆ alkyl” groups are methyl, ethyl and n-propyl.

The term “halogen” and “halo” are used interchangeably herein and denote fluoro, chloro, bromo, or iodo.

The term “heterocyclyl” denotes a monovalent saturated or partly unsaturated mono- or bicyclic ring system of 3 to 12 ring atoms, comprising 1, 2, or 3 ring heteroatoms selected from N, O and S, the remaining ring atoms being carbon. In particular embodiments, heterocyclyl is a monovalent saturated monocyclic ring system of 4 to 10 ring atoms, comprising 1, 2, or 3 ring heteroatoms selected from N, O and S, the remaining ring atoms being carbon. Heterocyclyl can be fully or partially saturated. Examples for monocyclic saturated heterocyclyl are aziridinyl, oxiranyl, azetidinyl, oxetanyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for partially saturated monocyclic heterocyclyl are dihydrofuryl, imidazolinyl, dihydro-oxazolyl, tetrahydropyridinyl, and dihydropyranyl. Examples for bicyclic heterocyclyl are 1,3-dihydropyrrolo[3,4-c]pyridinyl; 2,3,3a,7a-tetrahydro-1H-indenylamino; 3,4-dihydro-1H-isoquinolinyl; 5,7-dihydropyrrolo[3,4-b]pyridinyl; 5,7-dihydropyrrolo[3,4-d]pyrimidinyl; isoindolinyl; azabicyclo[3.2.1]octanyl; azabicyclo[3.3.1]nonanyl; azaspiro[3.3]heptanyl; oxaazabicyclo[3.3.1]nonanyl; oxadiazaspiro[4.5]decanyl; diazabicyclo[2.2.2]octanyl; diazabicyclo[3.2.1]octanyl; diazabicyclo[4.2.0]octanyl; diazaspiro[2.5]octanyl; diazaspiro[3.3]heptanyl; diazaspiro[3.4]octanyl; diazaspiro[3.5]nonanyl; diazaspiro[3.6]decanyl; diazaspiro[4.4]nonanyl; diazaspiro[4.5]decanyl; diazaspiro[5.5]undecanyl; oxadiazabicyclo[3.3.1]nonanyl; oxadiazaspiro[5.5]undecanyl; and oxodiazaspiro[4.4]nonanyl. Monocyclic or bicyclic heterocyclyl can be further substituted by halogen, hydroxy, amino, aminoC₁₋₆ alkyl, aminoC₁₋₆alkylcarbonyl, C₁₋₆ alkylcarbonylamino, (C₁₋₆alkyl)₂amino, carbamoyl, C₁₋₆alkyl, haloC₁₋₆ alkyl, phenyl, phenylC₁₋₆ alkyl, or heterocyclyl.

The term “enantiomer” denotes two stereoisomers of a compound which are non-superimposable mirror images of one another.

The term “diastereomer” denotes a stereoisomer with two or more centers of chirality and whose molecules are not mirror images of one another. Diastereomers have different physical properties, e.g. melting points, boiling points, spectral properties, and reactivities.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

Antagonist of TLR7 and/or TLR8 and/or TLR9
The present invention relates to a compound of formula (I),

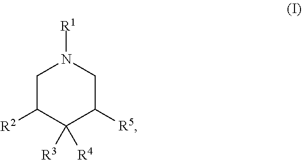

wherein

### R¹ is

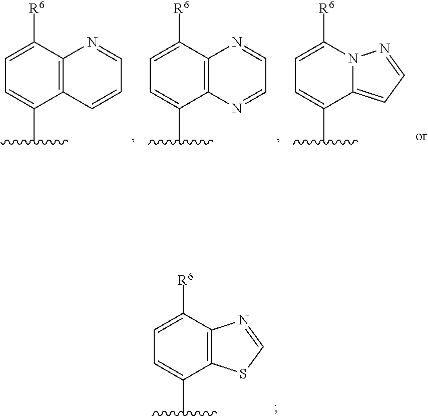

wherein R⁶ is cyano or halogen;
R² is C₁₋₆alkyl;
R³ is H or halogen;
R⁴ is H, halogen or hydroxy;
R⁵ is 1,3-dihydropyrrolo[3,4-c]pyridinyl substituted by piperazinyl;

2,3,3a,7a-tetrahydro-1H-indenylamino substituted by piperazinyl;

3,4-dihydro-1H-isoquinolinyl substituted by piperazinyl;

5,7-dihydropyrrolo[3,4-b]pyridinyl substituted by piperazinyl;

5,7-dihydropyrrolo[3,4-d]pyrimidinyl substituted by piperazinyl;

isoindolinyl substituted by piperazinyl; or

—NR^{5a}R^{5b}; wherein
R^{5a} is H or C₁₋₆alkyl;
R^{5b} is 1,2,3,4-tetrahydroisoquinolinylC₁₋₆ alkyl;
   5,6,7,8-tetrahydro-1,6-naphthyridinylC₁₋₆alkyl;
   isoindolinylC₁₋₆alkyl;
   phenyl(hydroxy)C₁₋₆ alkyl substituted by piperazinyl;
   phenylC₁₋₆alkyl, said phenylC₁₋₆alkyl being substituted by one or two substituents independently selected from (C₁₋₆ alkyl)₂pyridinyl, aminopiperidinyl, aminopyrrolidinyl, C₁₋₆alkylpiperazinyl, halogen, piperazinyl and piperidinyl;
   pyrazinylC₁₋₆ alkyl substituted by piperazinyl;
   pyridazinylC₁₋₆ alkyl substituted by piperazinyl;
   pyridinylC₁₋₆ alkyl substituted by piperazinyl or C₁₋₆alkylpiperazinyl; or
   pyrimidinylC₁₋₆ alkyl substituted by piperazinyl or C₁₋₆alkylpiperazinyl;
      or a pharmaceutically acceptable salt, enantiomer or diastereomer thereof.

A further embodiment of present invention is (ii) a compound of formula (Ia),

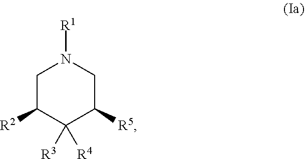

wherein

### R¹ is

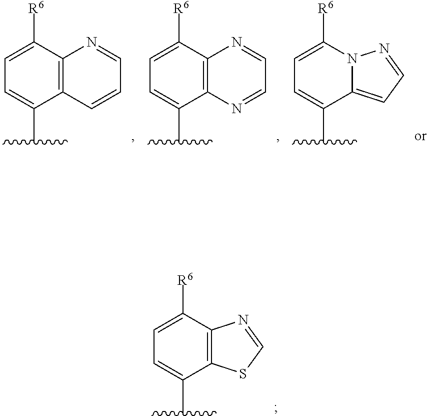

wherein R⁶ is cyano or halogen;
R² is C₁₋₆alkyl;
R³ is H or halogen;
R⁴ is H, halogen or hydroxy;
R⁵ is 1,3-dihydropyrrolo[3,4-c]pyridinyl substituted by piperazinyl;

2,3,3a,7a-tetrahydro-1H-indenylamino substituted by piperazinyl;

3,4-dihydro-1H-isoquinolinyl substituted by piperazinyl;

5,7-dihydropyrrolo[3,4-b]pyridinyl substituted by piperazinyl;

5,7-dihydropyrrolo[3,4-d]pyrimidinyl substituted by piperazinyl;

isoindolinyl substituted by piperazinyl; or

—NR^{5a}R^{5b}; wherein
R^{5a} is H or C₁₋₆alkyl;
R^{5b} is 1,2,3,4-tetrahydroisoquinolinylC₁₋₆ alkyl;
   5,6,7,8-tetrahydro-1,6-naphthyridinylC₁₋₆alkyl;
   isoindolinylC₁₋₆alkyl;
   phenyl(hydroxy)C₁₋₆ alkyl substituted by piperazinyl;
   phenylC₁₋₆alkyl, said phenylC₁₋₆alkyl being substituted by one or two substituents independently selected from (C₁₋₆ alkyl)₂pyridinyl, aminopiperidinyl, aminopyrrolidinyl, C₁₋₆alkylpiperazinyl, halogen, piperazinyl and piperidinyl;
   pyrazinylC₁₋₆ alkyl substituted by piperazinyl;
   pyridazinylC₁₋₆ alkyl substituted by piperazinyl;
   pyridinylC₁₋₆ alkyl substituted by piperazinyl or C₁₋₆alkylpiperazinyl; or
   pyrimidinylC₁₋₆ alkyl substituted by piperazinyl or C₁₋₆alkylpiperazinyl;
      or a pharmaceutically acceptable salt, enantiomer or diastereomer thereof.

A further embodiment of present invention is (iii) a compound of formula (I) or (Ia) according to (i) or (ii), wherein

### R¹ is

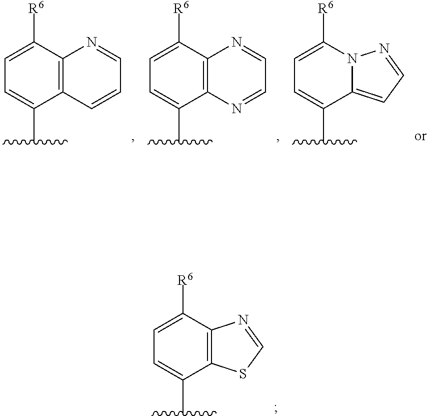

wherein R⁶ is cyano or halogen;
R² is C₁₋₆alkyl;

### R³ is H;

### R⁴ is H;

R⁵ is 3,4-dihydro-1H-isoquinolinyl substituted by piperazinyl;

5,7-dihydropyrrolo[3,4-b]pyridinyl substituted by piperazinyl;

isoindolinyl substituted by piperazinyl; or

—NR^{5a}R^{5b}; wherein
R^{5a} is H or C₁₋₆alkyl;
R^{5b} is 1,2,3,4-tetrahydroisoquinolinylC₁₋₆ alkyl;
   5,6,7,8-tetrahydro-1,6-naphthyridinylC₁₋₆alkyl;
   phenylC₁₋₆alkyl, said phenylC₁₋₆alkyl being substituted by one or two substituents independently selected from (C₁₋₆ alkyl)₂pyridinyl, aminopiperidinyl, aminopyrrolidinyl, C₁₋₆alkylpiperazinyl, halogen, piperazinyl and piperidinyl;
   pyrazinylC₁₋₆ alkyl substituted by piperazinyl;
   pyridinylC₁₋₆ alkyl substituted by piperazinyl or C₁₋₆alkylpiperazinyl; or
   pyrimidinylC₁₋₆ alkyl substituted by piperazinyl or C₁₋₆alkylpiperazinyl;

or a pharmaceutically acceptable salt, enantiomer or diastereomer thereof.

A further embodiment of present invention is (iv) a compound of formula (I) or (Ia) according to (iii), wherein

### R¹ is

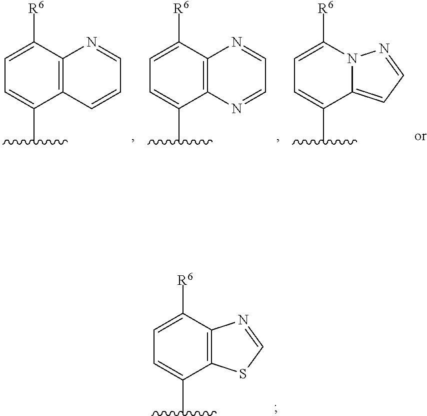

wherein R⁶ is cyano or chloro;
R² is methyl;

### R³ is H;

### R⁴ is H;

R⁵ is ((aminopiperidinyl)phenyl)methylamino; ((aminopyrrolidinyl)phenyl)methylamino; ((dimethylpyridinyl)phenyl)methylamino; ((methylpiperazinyl)phenyl)methylamino; ((methylpiperazinyl)pyridinyl)methylamino; ((methylpiperazinyl)pyrimidinyl)methylamino; (5,6,7,8-tetrahydro-1,6-naphthyridinyl)methylamino; (fluoro(piperazinyl)phenyl)methylamino; (piperazinylphenyl)ethylamino; (piperazinylphenyl)methylamino; (piperazinylpyrazinyl)methylamino; (piperazinylpyridinyl)methylamino; (piperazinylpyrimidinyl)methylamino; (piperidinylphenyl)methylamino; 1,2,3,4-tetrahydroisoquinolinylmethylamino; methyl((piperazinylphenyl)methyl)amino; piperazinyl-3,4-dihydro-1H-isoquinolinyl; piperazinyl-5,7-dihydropyrrolo[3,4-b]pyridinyl; or piperazinylisoindolinyl;

or a pharmaceutically acceptable salt, enantiomer or diastereomer thereof.

A further embodiment of present invention is (v) a compound of formula (I) or (Ia) according to (iii), wherein A compound according to claim 3, wherein R¹ is

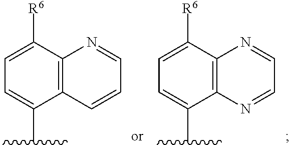

wherein R⁶ is cyano.

A further embodiment of present invention is (vi) a compound of formula (I) or (Ia) according to (v), wherein R² is methyl.

A further embodiment of present invention is (vii) a compound of formula (I) or (Ia) according to (vi), wherein

R⁵ is 5,7-dihydropyrrolo[3,4-b]pyridinyl substituted by piperazinyl; or

—NR^{5a}R^{5b}; wherein
R^{5a} is H;
R^{5b} is phenylC₁₋₆ alkyl substituted by piperazinyl; or pyrimidinylC₁₋₆ alkyl substituted by piperazinyl.

A further embodiment of present invention is (viii) a compound of formula (I) or (Ia) according to (vii), wherein R⁵ is piperazinyl-5,7-dihydropyrrolo[3,4-b]pyridinyl; (piperazinylphenyl)methylamino or (piperazinylpyrimidinyl)methylamino.

A further embodiment of present invention is (ix) a compound of formula (I) or (Ia) according to (i) or (ii), wherein

### R¹ is

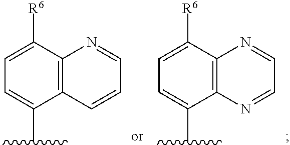

wherein R⁶ is cyano;
R² is C₁₋₆alkyl;

### R³ is H;

### R⁴ is H;

R⁵ is R⁵ is 5,7-dihydropyrrolo[3,4-b]pyridinyl substituted by piperazinyl; or

—NR^{5a}R^{5b}; wherein
R^{5a} is H;
R^{5b} is phenylC₁₋₆ alkyl substituted by piperazinyl; or pyrimidinylC₁₋₆ alkyl substituted by piperazinyl;
   or a pharmaceutically acceptable salt, enantiomer or diastereomer thereof.

A further embodiment of present invention is (x) a compound of formula (I) or (Ia) according to (ix), wherein

### R¹ is

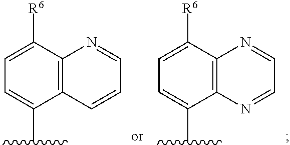

wherein R⁶ is cyano;
R² is methyl;

### R³ is H;

### R⁴ is H;

R⁵ is piperazinyl-5,7-dihydropyrrolo[3,4-b]pyridinyl; (piperazinylphenyl)methylamino or (piperazinylpyrimidinyl)methylamino;
or a pharmaceutically acceptable salt, enantiomer or diastereomer thereof.

Another embodiment of present invention is that (xi) particular compounds of formula (I) or (Ia) are the following:
5-[(3S,5R)-3-methyl-5-[(4-piperazin-1-ylphenyl)methylamino]-1-piperidyl]quinoline-8-carbonitrile;
5-[(3S,5R)-3-methyl-5-[[4-(4-piperidyl)phenyl]methylamino]-1-piperidyl]quinoline-8-carbonitrile;
8-[(3S,5R)-3-methyl-5-[(4-piperazin-1-ylphenyl)methylamino]-1-piperidyl]quinoxaline-5-carbonitrile;
5-[(3R,5S)-3-[(2-fluoro-4-piperazin-1-yl-phenyl)methylamino]-5-methyl-1-piperidyl]quinoline-8-carbonitrile;
5-[(3S,5R)-3-methyl-5-[(5-piperazin-1-yl-2-pyridyl)methylamino]-1-piperidyl]quinoline-8-carbonitrile;
5-[(3S,5R)-3-methyl-5-[[3-(4-methylpiperazin-1-yl)phenyl]methylamino]-1-piperidyl]quinoline-8-carbonitrile;
5-[(3S,5R)-3-methyl-5-[[4-(4-methylpiperazin-1-yl)phenyl]methylamino]-1-piperidyl]quinoline-8-carbonitrile;
5-[(3S,5R)-3-methyl-5-[(3-piperazin-1-ylphenyl)methylamino]-1-piperidyl]quinoline-8-carbonitrile;
5-[(3S,5R)-3-methyl-5-[methyl-[(4-piperazin-1-ylphenyl)methyl]amino]-1-piperidyl]quinoline-8-carbonitrile;
5-[(3 S, 5R)-3-methyl-5-[methyl-[(4-piperazin-1-ylphenyl)methyl]amino]-1-piperidyl]quinoline-8-carbonitrile;
5-[(3S,5R)-3-methyl-5-(1,2,3,4-tetrahydroisoquinolin-6-ylmethylamino)-1-piperidyl]quinoline-8-carbonitrile;
5-[(3S,5R)-3-methyl-5-(5-piperazin-1-ylisoindolin-2-yl)-1-piperidyl]quinoline-8-carbonitrile;
5-[(3R,5 S)-3-[[4-(3-aminopyrrolidin-1-yl)phenyl]methylamino]-5-methyl-1-piperidyl]quinoline-8-carbonitrile;
5-[(3R,5 S)-3-[[4-(3-amino-1-piperidyl)phenyl]methylamino]-5-methyl-1-piperidyl]quinoline-8-carbonitrile;
5-[(3R,5S)-3-[[4-(2,6-dimethyl-4-pyridyl)phenyl]methylamino]-5-methyl-1-piperidyl]quinoline-8-carbonitrile;
5-[(3S,5R)-3-methyl-5-[[2-(4-methylpiperazin-1-yl)pyrimidin-5-yl]methylamino]-1-piperidyl]quinoline-8-carbonitrile;
5-[(3S,5R)-3-methyl-5-[(2-piperazin-1-ylpyrimidin-5-yl)methyl amino]-1-piperidyl]quinoline-8-carbonitrile;
7-[(3S,5R)-3-methyl-5-[[5-(4-methylpiperazin-1-yl)-2-pyridyl]methylamino]-1-piperidyl]-1,3-benzothiazole-4-carbonitrile;
4-[(3S,5R)-3-methyl-5-[(4-piperazin-1-ylphenyl)methylamino]-1-piperidyl]pyrazolo[1,5-a]pyridine-7-carbonitrile;
(3R,5S)-1-(8-chloro-5-quinolyl)-5-methyl-N-[(4-piperazin-1-ylphenyl)methyl]piperidin-3-amine;
5-[(3S,5R)-3-methyl-5-(3-piperazin-1-yl-5,7-dihydropyrrolo[3,4-b]pyridin-6-yl)-1-piperidyl]quinoline-8-carbonitrile;
5-[cis-3-methyl-5-(6-piperazin-1-yl-3,4-dihydro-1H-isoquinolin-2-yl)-1-piperidyl]quinoline-8-carbonitrile;
5-[(3S,5R)-3-methyl-5-[(5-piperazin-1-ylpyrimidin-2-yl)methyl amino]-1-piperidyl]quinoline-8-carbonitrile;
5-[(3 S, 5R)-3-methyl-5-[1-(4-piperazin-1-ylphenyl)ethylamino]-1-piperidyl]quinoline-8-carbonitrile;
5-[(3S,5R)-3-methyl-5-[[2-[(3R)-3-methylpiperazin-1-yl]pyrimidin-5-yl]methylamino]-1-piperidyl]quinoline-8-carbonitrile;
5-[(3S,5R)-3-methyl-5-[[2-[(3S)-3-methylpiperazin-1-yl]pyrimidin-5-yl]methylamino]-1-piperidyl]quinoline-8-carbonitrile;
5-[(3S,5R)-3-methyl-5-(5,6,7,8-tetrahydro-1,6-naphthyridin-2-ylmethylamino)-1-piperidyl]quinoline-8-carbonitrile; and
5-[(3S,5R)-3-methyl-5-[(5-piperazin-1-ylpyrazin-2-yl)methylamino]-1-piperidyl]quinoline-8-carbonitrile;

or a pharmaceutically acceptable salt, enantiomer or diastereomer thereof.

A number of compounds were disclosed in patent WO2015057655 showing TLR7 and TLR9 potency data summarized in table 1. From the chemical structure perspective, compounds in table 1 are characterized in having structures as shown in formula (A). More specifically, piperidine ring is substituted by a linear basic center (amino group) connected to aryl or heteroaryl in the tail part. According to the potency data disclosed in patent WO2015057655, compounds of formula (A) only showed good TLR7 potency, but suffered from the loss of TLR9 potency.

Meanwhile, more analogues of the compounds disclosed in WO2015057655, such as compound R1 and compound R2 which are even closer to the compounds of this invention, were synthesized to confirm the SAR (structure-activity-relationship). However, according to the potency data shown in Table 2, the structural modification based on formula (A) still could not improve the potency of TLR9. Therefore, the skill of the art shall not obtain any incitation from the information disclosed in WO2015057655 to further optimize such chemical structures.

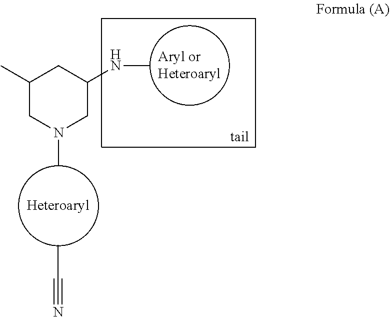

Surprisingly, the compounds of this invention significantly improved TLR9 potency (>10 folds compared to ER-899552) while keeping excellent TLR7 and TLR8 potency. In another embodiment, the human microsome stability of the compounds of this invention was greatly increased compared to the reference compound R¹ and R² (see Table 3).

TABLE 1
TLR7 and TLR9 potency of compounds disclosed in WO2015057655

| | | HEK/hTLR7 | HEK/hTLR9 |
|---|---|---|---|
| Compound | Structure | IC50 (μM) | IC50 (μM) |
| ER-899543 | 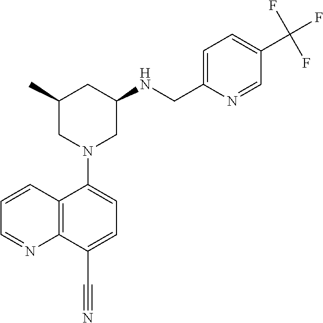 [SMILES: C[C@H]1C[C@@H](NCc2ccc(C(F)(F)F)cn2)CN(c2ccc(C#N)c3ncccc23)C1] | 0.0480 | >10.0 |
| ER-899551 | 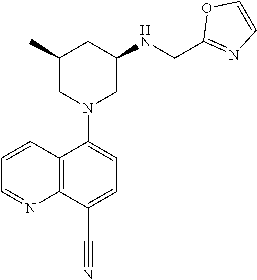 [SMILES: C[C@H]1C[C@@H](NCc2ncco2)CN(c2ccc(C#N)c3ncccc23)C1] | 0.0430 | >10.0 |
| ER-899552 | 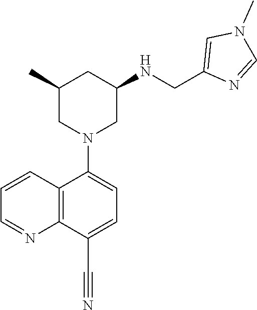 [SMILES: C[C@H]1C[C@@H](NCc2cn(C)cn2)CN(c2ccc(C#N)c3ncccc23)C1] | 0.2070 | 1.969 |
| ER-899607 | 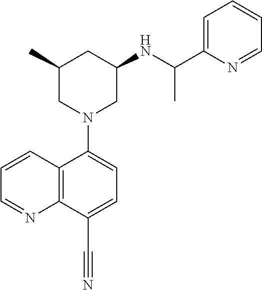 [SMILES: CC(N[C@@H]1C[C@H](C)CN(c2ccc(C#N)c3ncccc23)C1)c1ccccn1] | 0.1489 | >10.0 |

Reference compounds R1 and R2 were synthesized according to the method described in this invention.

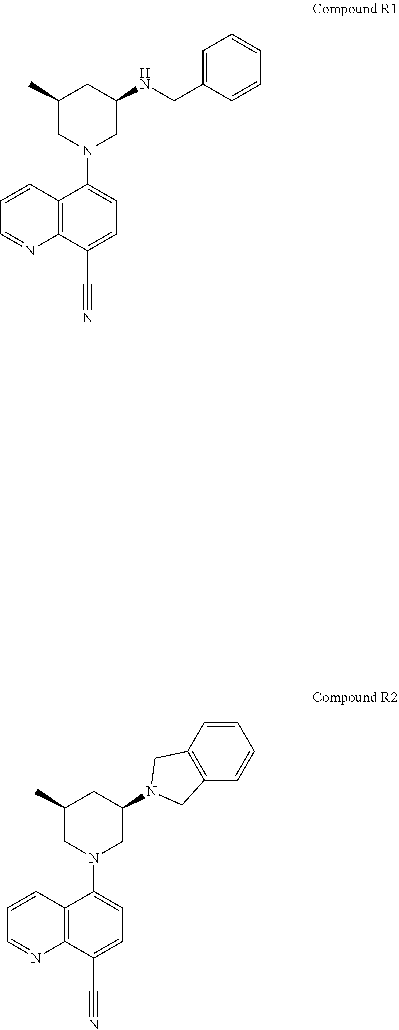 [SMILES: C[C@H]1C[C@@H](NCc2ccccc2)CN(c2ccc(C#N)c3ncccc23)C1 ; C[C@H]1C[C@@H](N2Cc3ccccc3C2)CN(c2ccc(C#N)c3ncccc23)C1]

### Synthesis

The compounds of the present invention can be prepared by any conventional means. Suitable processes for synthesizing these compounds as well as their starting materials are provided in the schemes below and in the examples. All substituents, in particular, R¹ to R⁵ are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I), (Ia), (VIII), (XI), (XII), (XIV) or (XX) are shown below.

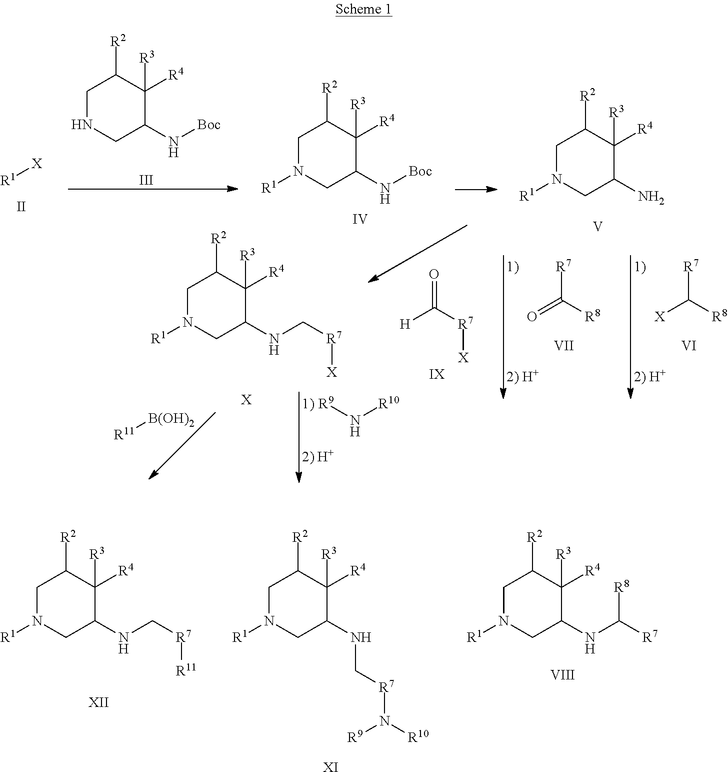

wherein X is halogen; R⁷ is heterocyclyl; R⁸ is C₁₋₆ alkyl or hydroxyC₁₋₆alkyl; R⁹ and R¹⁰ together with the carbon atoms they are attached to form a heterocyclyl; R¹¹ is aryl or heteroaryl.

The synthesis of compounds of the present invention started from halide II. Buchwald-Hartwig amination reaction between halide II and compound of formula III provides compound of formula IV with a catalyst, such as Ruphos Pd-G2, and a base, such as Cs₂CO₃ (ref: Acc. Chem. Res. 1998, 31, 805-818; Chem. Rev. 2016, 116, 12564-12649; Topics in Current Chemistry, 2002, 219, 131-209; and references cited therein). Deprotection of compound of formula IV in acidic condition, such as HCl in EtOAc and TFA in DCM, gives compound V, which can be transformed into compound of formula VIII either via nucleophilic substitution with compound of formula VI in the presence of a base, such as DIPEA and K₂CO₃, or via reductive amination with compound of formula VII followed by appropriate deprotection. Alternatively, compound of formula V can react with aldehyde IX to give compound of formula X. Buchwald-Hartwig amination reaction between compound of formula X and an amine provides compound of formula XI that may bear a suitable protecting group, e.g. Boc, which can be removed in acidic conditions to afford the final compound. Palladium catalyzed Suzuki-Miyaura reaction between compound of formula X and a boronic acid R¹¹—B(OH)₂ provides compound of formula XII.

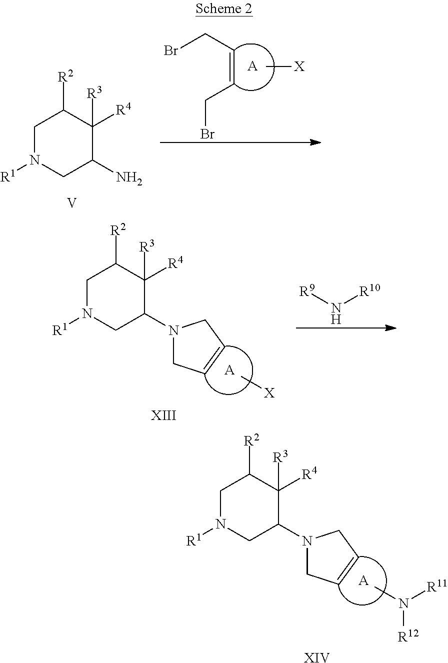

wherein ring A is heterocyclyl; R⁹ and R¹⁰ are as defined above.

Compound of formula V can react with a dibromide compound in the presence of a base, such as DIPEA and K₂CO₃, to give compound of formula XIII Buchwald-Hartwig amination reaction between XIII and an amine provides compound of formula XIV that may bear a suitable protecting group, e.g. Boc, which can be removed in acidic conditions to afford the final compound.

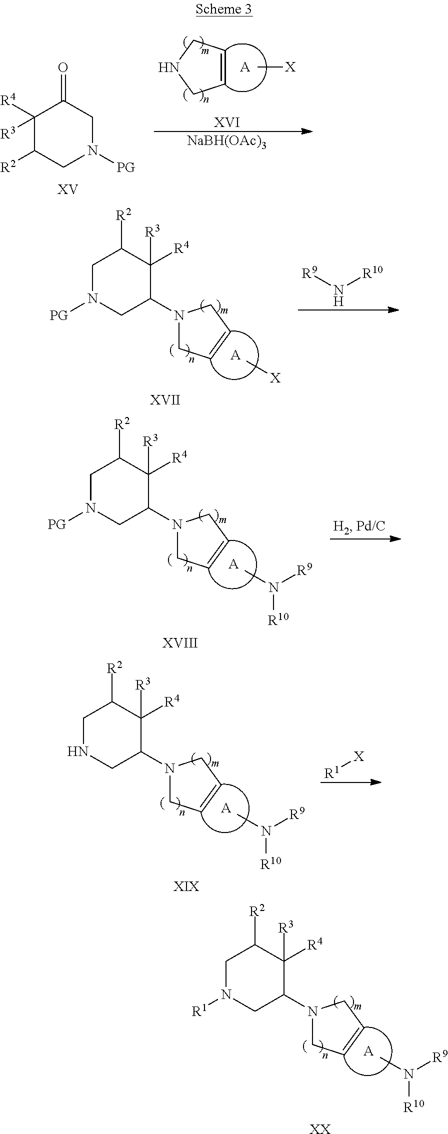

wherein PG is protecting group, such as Bn and Cbz; m is 1 or 2; n is 1 or 2; ring A, R¹¹ and R¹² are as defined above.

Reductive amination between compound of formula XV and XVI affords compound of XVII, which is then coupled with R¹¹R¹²NH under Buchwald-Hartwig amination conditions to give compound of formula XVIII Subsequent deprotection of compound of formula XVIII in the presence of H₂ provides compound of formula XIX. Buchwald-Hartwig amination reaction between compound of formula XIX and halide II as described above affords compound of formula XX that may bear a suitable protecting group, e.g. Boc, which can be removed in acidic conditions to afford the final compound.

This invention also relates to a process for the preparation of a compound of formula (I) or (Ia) comprising any of the following steps:

a) the nucleophilic substitution of compound of formula (V),

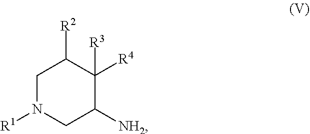

with compound (VI) in the presence of a base;

b) the reductive amination of compound of formula (V),

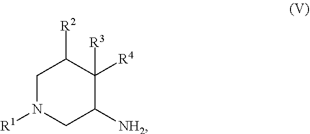

with compound (VII);

c) the Buchwald-Hartwig amination of compound of formula (X),

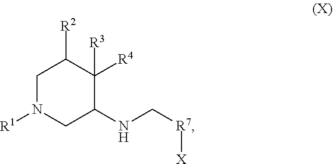

with an amine;

d) the Suzuki-Miyaura reaction of compound of formula (X),

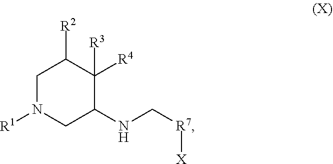

with an boronic acid;

e) the Buchwald-Hartwig amination of compound of formula (XIII),

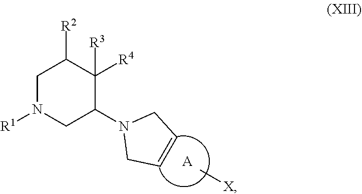

with an amine;

f) the Buchwald-Hartwig amination of compound of formula (XIX),

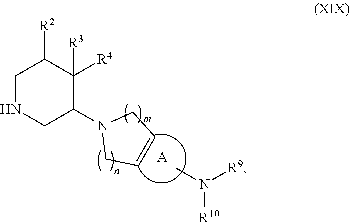

with halide (II);

In step a) the base can be for example K₂CO₃ or DIPEA.

A compound of formula (I) or (Ia) when manufactured according to the above process is also an object of the invention.

Compounds of this invention can be obtained as mixtures of diastereomers or enantiomers, which can be separated by methods well known in the art, e.g. (chiral) HPLC or SFC.

### Indications and Methods of Treatment

The present invention provides compounds that can be used as TLR7 and/or TLR8 and/or TLR9 antagonist, which inhibits pathway activation through TLR7 and/or TLR8 and/or TLR9 as well as respective downstream biological events including, but not limited to, innate and adaptive immune responses mediated through the production of all types of cytokines and all forms of auto-antibodies. Accordingly, the compounds of the invention are useful for blocking TLR7 and/or TLR8 and/or TLR9 in all types of cells that express such receptor(s) including, but not limited to, plasmacytoid dendritic cell, B cell, T cell, macrophage, monocyte, neutrophil, keratinocyte, epithelial cell. As such, the compounds can be used as a therapeutic or prophylactic agent for systemic lupus erythematosus and lupus nephritis.

The present invention provides methods for treatment or prophylaxis of systemic lupus erythematosus and lupus nephritis in a patient in need thereof.

Another embodiment includes a method of treating or preventing systemic lupus erythematosus and lupus nephritis in a mammal in need of such treatment, wherein the method comprises administering to said mammal a therapeutically effective amount of a compound of formula (I), a stereoisomer, tautomer, prodrug or pharmaceutically acceptable salt thereof.

### EXAMPLES

The invention will be more fully understood by reference to the following examples. They should not, however, be construed as limiting the scope of the invention.

### ABBREVIATIONS

The invention will be more fully understood by reference to the following examples. They should not, however, be construed as limiting the scope of the invention.

Abbreviations used herein are as follows:
DCE: dichloroethane
DIPEA or DIEA: N,N-diisopropylethylamine
DIBAL-H: Diisobutylaluminium hydride
DMA: N,N-Dimethylacetylamine
DMAP: 4-dimethylaminopyridine
DMF: N,N-Dimethylformamide
DPPP: 1,3-Bis(diphenylphosphino)propane
EA or EtOAc: ethyl acetate
FA: formic acid
HATU: 1-[Bis(dimethylamino)methylene]-1H-1,2,3-triazolo[4,5-b]pyridinium 3-oxid hexafluorophosphate
IC₅₀: half inhibition concentration
IPA: isopropanol
LCMS liquid chromatography-mass spectrometry
MS: mass spectrometry
NBS: N-bromosuccinimide
PE: petroleum ether
prep-HPLC: preparative high performance liquid chromatography
rt: room temperature
RT: retention time
RuPhos Pd G2: chloro(2-dicyclohexylphosphino-2′,6′-diisopropoxy-1,1′-biphenyl)[2-(2′-amino-1,1′-biphenyl)]palladium(II) 2nd generation
SFC: supercritical fluid chromatography
TFA: trifluoroacetic acid
TLC: thin layer chromatography
v/v volume ratio
LYSA lyophilisation solubility assay
HLM human liver microsome

General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C₁₈ (5 μm, OBD™ 30×100 mm) column, SunFire™ Prep-C₁₈ (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃.H₂O in MeOH), back pressure 100 bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):

Acidic condition I: A: 0.1% TFA in H₂O; B: 0.1% TFA in acetonitrile;

Acidic condition II: A: 0.0375% TFA in H₂O; B: 0.01875% TFA in acetonitrile;

Basic condition I: A: 0.1% NH₃.H₂O in H₂O; B: acetonitrile;

Basic condition II: A: 0.025% NH₃.H₂O in H₂O; B: acetonitrile;

Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### Preparative Examples

The following examples are intended to illustrate the meaning of the present invention but should by no means represent a limitation within the meaning of the present invention:

### Example 1

### 5-[(3S,5R)-3-methyl-5-[(4-piperazin-1-ylphenyl)methylamino]1-piperidyl]quinoline-8-carbonitrile

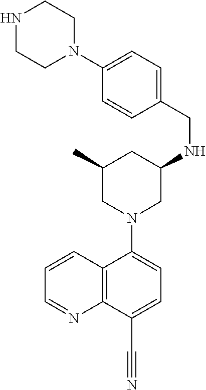 [SMILES: C[C@H]1C[C@@H](NCc2ccc(N3CCNCC3)cc2)CN(c2ccc(C#N)c3ncccc23)C1]

The title compound was prepared according to the following scheme:

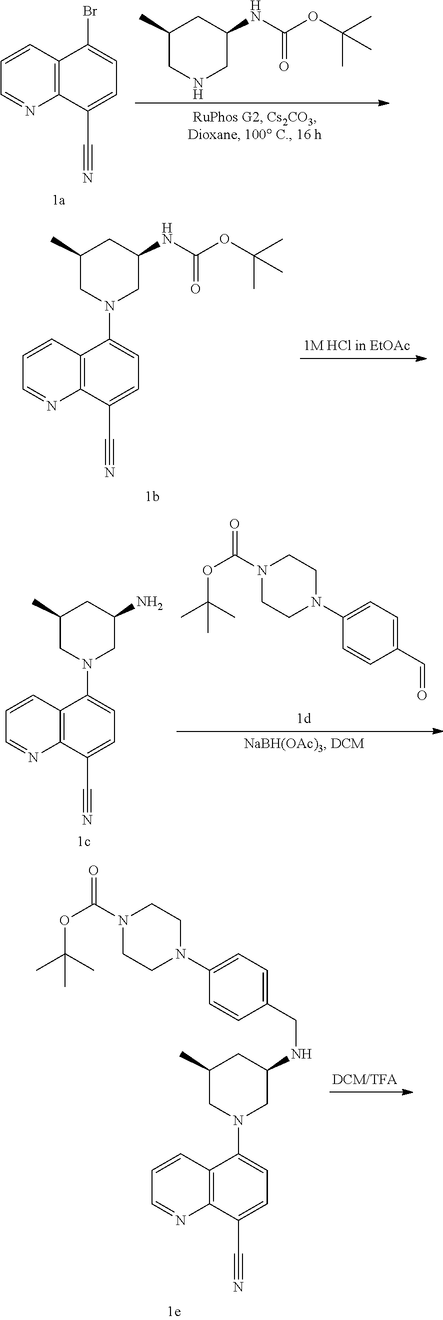

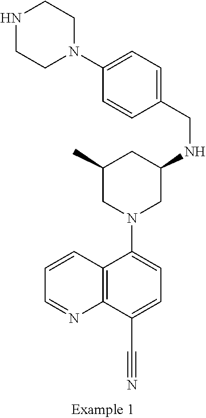 [SMILES: C[C@H]1C[C@@H](NCc2ccc(N3CCNCC3)cc2)CN(c2ccc(C#N)c3ncccc23)C1]

### Step 1: Preparation of tert-butyl N-[(3R,5S)-1-(8-cyano-5-quinolyl)-5-methyl-3-piperidyl]carbamate (Compound 1b)

A mixture of 5-bromoquinoline-8-carbonitrile (compound 1a, 400 mg, 1.72 mmol), tert-butyl N-[(3R,5S)-5-methyl-3-piperidyl]carbamate (368 mg, 1.72 mmol) (Reference: WO2015/057655), RuPhos G2 (133 mg, 172 μmol) and Cs₂CO₃ (839 mg, 2.57 mmol) in dioxane (20 mL) was charged with N₂, and heated to 80° C. overnight. After cooling, the solid was filtered off and washed with EtOAc (20 mL). The combined filtrate was concentrated. The residue was purified by chromatography (eluting with EtOAc in PE (10%-50%) to afford compound 1b as a light yellow solid. 250 mg, LCMS(M+H)⁺: 367.

### Step 2: Preparation of 5-[(3R,5S)-3-amino-5-methyl-1-piperidyl]quinoline-8-carbonitrile hydrochloride (Compound 1c)

A mixture of tert-butyl ((3R,5 S)-1-(8-cyanoquinolin-5-yl)-5-methylpiperidin-3-yl)carbamate (compound 1b, 250 mg, 682 μmol) in 1 M HCl in EtOAc (15 mL) was stirred at 23° C. for 16 hours, then the mixture was filtered to give compound 1c as an orange solid. 200 mg, LCMS(M+H)⁺: 267.

### Step 3: Preparation of tert-butyl 4-[4-[[[(3R,5S)-1-(8-cyano-5-quinolyl)-5-methyl-3-piperidyl]amino]methyl]phenyl]piperazine-1-carboxylate (Compound 1e)

A mixture of 5-((3R,5S)-3-amino-5-methylpiperidin-1-yl)quinoline-8-carbonitrile (compound 1c, 50 mg, 188 μmol), tert-butyl 4-(4-formylphenyl)piperazine-1-carboxylate (compound 1d, 109 mg, 375 μmol, Et₃N (26.2 μL, 188 μmol), AcOH (10.7 μL, 188 μmol) and sodium triacetoxyborohydride (119 mg, 563 μmol in DCM (10 mL) was stirred at 23° C. for 2 hours. The undissolved material was filtered off and the filtrate was concentrated and purified by prep-HPLC to give compound 1e as light yellow solid. 15 mg, LCMS (M+H)⁺: 541.

### Step 4: Preparation of 5-[(3S,5R)-3-methyl-5-[(4-piperazin-1-ylphenyl)methylamino]-1-piperidyl]quinoline-8-carbonitrile (Example 1)

A mixture of tert-butyl 4-(4-((((3R,5S)-1-(8-cyanoquinolin-5-yl)-5-methylpiperidin-3-yl)amino)methyl)phenyl)piperazine-1-carboxylate (compound 1e, 15 mg, 25.9 μmol) in DCM/TFA (2:1) (2 mL) was stirred at 23° C. for 1 hour, then the mixture was concentrated. The residue was dissolved in water and then lyophilized to give Example 1 as a brown solid (18 mg). LCMS(M+H)⁺: 441, ¹H NMR (400 MHz, METHANOL-d₄) δ ppm: 9.05 (dd, J=1.6, 4.3 Hz, 1H), 8.65 (dd, J=1.7, 8.6 Hz, 1H), 8.22 (d, J=8.1 Hz, 1H), 7.74 (dd, J=4.4, 8.6 Hz, 1H), 7.49 (d, J=8.7 Hz, 2H), 7.37 (d, J=8.1 Hz, 1H), 7.12 (d, J=8.7 Hz, 2H), 4.38-4.22 (m, 2H), 3.89 (br, 1H), 3.82-3.71 (m, 1H), 3.49 (dd, J=3.7, 6.4 Hz, 5H), 3.43-3.37 (m, 4H), 2.94 (t, J 11.0 Hz, 1H), 2.66-2.45 (m, 2H), 2.24 (br, 1H), 1.45-1.31 (m, 1H), 1.11 (d, J=6.6 Hz, 3H).

### Example 2

### 5-[(3S,5R)-3-methyl-5-[[4-(4-piperidyl)phenyl]methylamino]-1-piperidyl]quinoline-8-carbonitrile

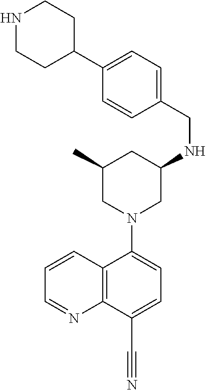 [SMILES: C[C@H]1C[C@@H](NCc2ccc(C3CCNCC3)cc2)CN(c2ccc(C#N)c3ncccc23)C1]

The title compound was prepared according to the following scheme:

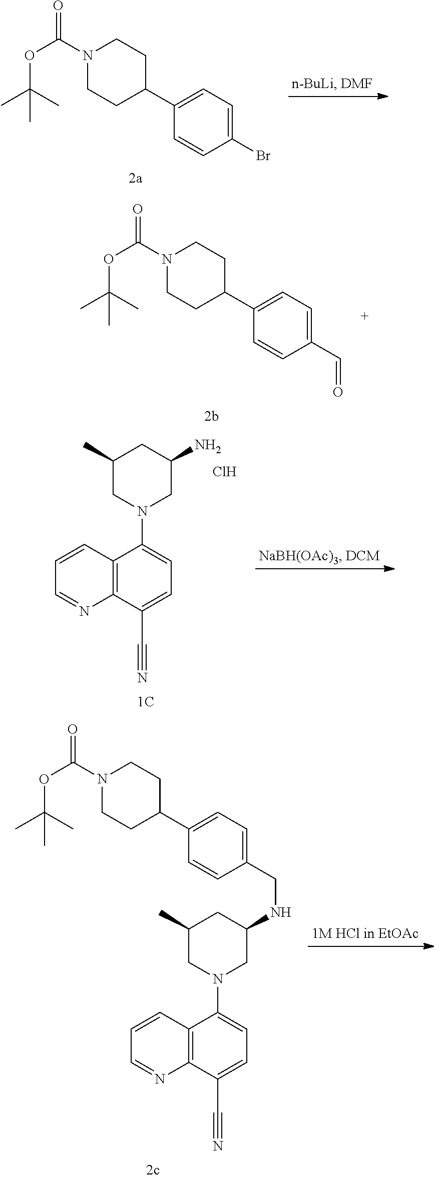 [SMILES: CC(C)(C)OC(=O)N1CCC(c2ccc(Br)cc2)CC1 ; CC(C)(C)OC(=O)N1CCC(c2ccc(C=O)cc2)CC1 ; C[C@H]1C[C@@H](N)CN(c2ccc(C#N)c3ncccc23)C1 ; Cl ; C[C@H]1C[C@@H](NCc2ccc(C3CCN(C(=O)OC(C)(C)C)CC3)cc2)CN(c2ccc(C#N)c3ncccc23)C1]

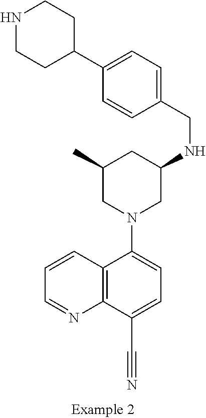 [SMILES: C[C@H]1C[C@@H](NCc2ccc(C3CCNCC3)cc2)CN(c2ccc(C#N)c3ncccc23)C1]

### Step 1: Preparation of tert-butyl 4-(4-formylphenyl)piperidine-1-carboxylate (Compound 2b)

To a solution of tert-butyl 4-(4-bromophenyl)piperidine-1-carboxylate (compound 2a, 340 mg, 1 mmol) in dry THF (20 mL) was added n-butyllithium (4.16 mL, 5 mmol) slowly at −78° C. After 10 mins, DMF (1 mL) was added and the mixture was stirred at rt for 30 mins. The reaction mixture was poured into 20 mL H₂O, then extracted with EtOAc. The organic layer was washed with water and brine, and then concentrated to give compound 2b as light yellow oil (250 mg), which was used in next step without further purification. LCMS (M+H)⁺: 290.

### Step 2: Preparation of 5-[(3S,5R)-3-methyl-5-[[4-(4-piperidyl)phenyl]methylamino]-1-piperidyl]quinoline-8-carbonitrile (Example 2)

The title compound was prepared in analogy to the preparation of Example 1 by using compound 2b instead of compound 1d. Example 2 was obtained as a light brown solid (14 mg). LCMS (M+H)⁺: 440, ¹H NMR (400 MHz, METHANOL-d₄) δ ppm: 9.03 (dd, J=1.7, 4.2 Hz, 1H), 8.58 (dd, J=1.6, 8.6 Hz, 1H), 8.19 (d, J=7.9 Hz, 1H), 7.70 (dd, J=4.2, 8.6 Hz, 1H), 7.53 (d, J=8.3 Hz, 2H), 7.41 (d, J=8.2 Hz, 2H), 7.34 (d, J=8.1 Hz, 1H), 4.36 (q, J=12.9 Hz, 2H), 3.86 (br, 1H), 3.78 (br, 1H), 3.60-3.46 (m, 3H), 3.23-3.09 (m, 3H), 3.05-2.85 (m, 2H), 2.63-2.46 (m, 2H), 2.24 (br, 1H), 2.07 (br, 2H), 1.99-1.84 (m, 2H), 1.12 (d, J=6.7 Hz, 3H)

### Example 3

### 8-[(3S,5R)-3-methyl-5-[(4-piperazin-1-ylphenyl)methylamino]-1-piperidyl]quinoxaline-5-carbonitrile

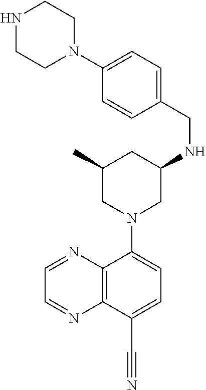 [SMILES: C[C@H]1C[C@@H](NCc2ccc(N3CCNCC3)cc2)CN(c2ccc(C#N)c3nccnc23)C1]

The title compound was prepared according to the following scheme:

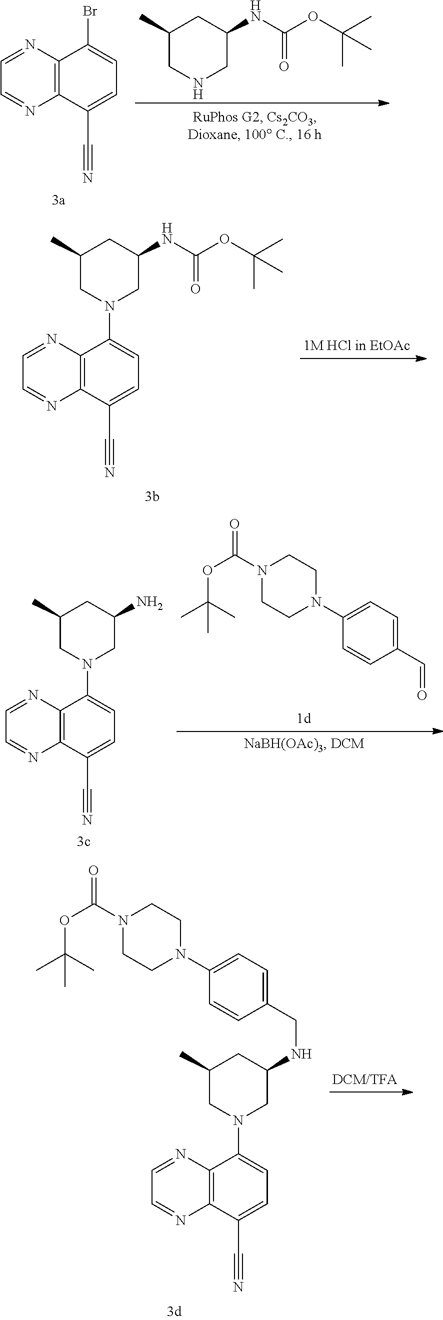

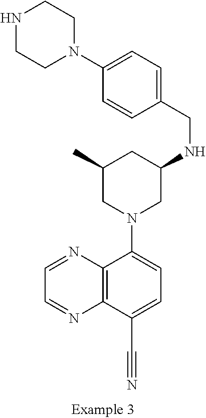 [SMILES: C[C@H]1C[C@@H](NCc2ccc(N3CCNCC3)cc2)CN(c2ccc(C#N)c3nccnc23)C1]

### Preparation of Example 3:

The title compound was prepared in analogy to the preparation of Example 1 by using 8-bromoquinoxaline-5-carbonitrile (compound 3a) (Reference: WO2017/106607) instead of compound 1a. Example 3 was obtained as a light brown solid (58 mg). LCMS(M+H)⁺: 442, ¹H NMR (400 MHz, METHANOL-d₄) δ ppm: 9.07-8.87 (m, 2H), 8.16 (d, J=8.3 Hz, 1H), 7.49 (d, J=8.7 Hz, 2H), 7.31 (d, J=8.4 Hz, 1H), 7.12 (d, J=8.8 Hz, 2H), 4.84 (td, J=1.7, 11.7 Hz, 1H), 4.39-4.22 (m, 2H), 4.03 (br, 1H), 3.74-3.63 (m, 1H), 3.52-3.45 (m, 4H), 3.43-3.35 (m, 4H), 3.00 (t, J=11.3 Hz, 1H), 2.74 (t, J=11.8 Hz, 1H), 2.46 (br d, J=11.9 Hz, 1H), 2.20-2.04 (m, 1H), 1.37 (q, J=11.8 Hz, 1H), 1.12 (d, J=6.6 Hz, 3H).

### Example 4

### 5-[(3R,58)-3-[(2-fluoro-4-piperazin-1-yl-phenyl)methylamino]-5-methyl-1-piperidyl]quinoline-8-carbonitrile

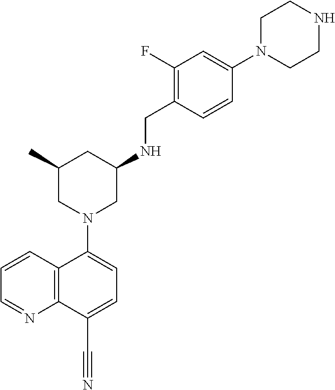 [SMILES: C[C@H]1C[C@@H](NCc2ccc(N3CCNCC3)cc2F)CN(c2ccc(C#N)c3ncccc23)C1]

The title compound was prepared according to the following scheme:

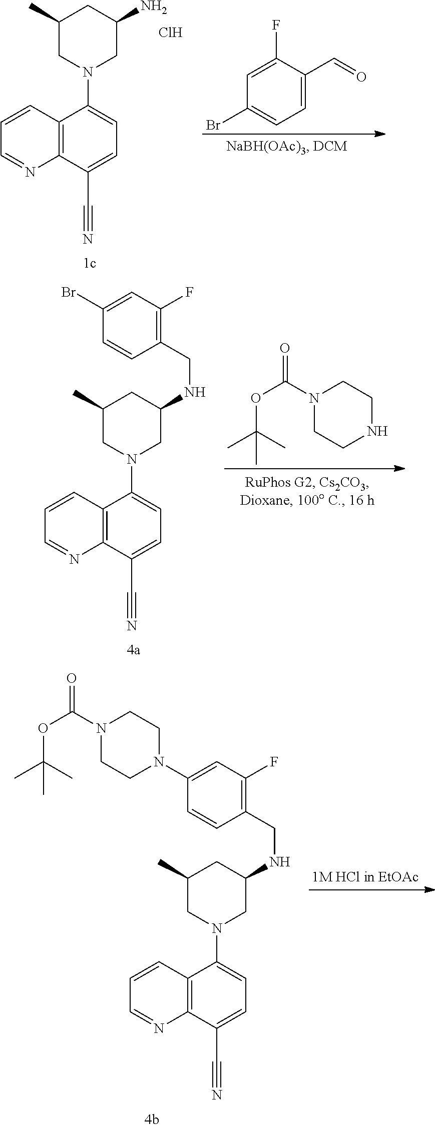 [SMILES: C[C@H]1C[C@@H](N)CN(c2ccc(C#N)c3ncccc23)C1 ; Cl ; O=Cc1ccc(Br)cc1F ; C[C@H]1C[C@@H](NCc2ccc(Br)cc2F)CN(c2ccc(C#N)c3ncccc23)C1 ; CC(C)(C)OC(=O)N1CCNCC1 ; C[C@H]1C[C@@H](NCc2ccc(N3CCN(C(=O)OC(C)(C)C)CC3)cc2F)CN(c2ccc(C#N)c3ncccc23)C1]

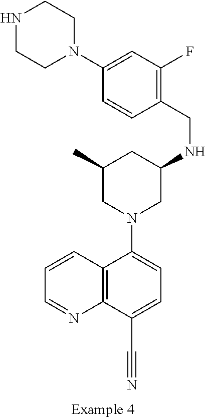 [SMILES: C[C@H]1C[C@@H](NCc2ccc(N3CCNCC3)cc2F)CN(c2ccc(C#N)c3ncccc23)C1]

### Step 1: Preparation of 5-((3R,5S)-3-((4-bromo-2-fluorobenzyl)amino)-5-methylpiperidin-1-yl)quinoline-8-carbonitrile (Compound 4a)

A mixture of 5-((3R,5S)-3-amino-5-methylpiperidin-1-yl)quinoline-8-carbonitrile hydrochloride (compound 1c, 60 mg, 198 μmol), 4-bromo-2-fluorobenzaldehyde (40.2 mg, 198 μmol, Et₃N (55.2 μL, 396 μmol), acetic acid (594 μmol) and sodium triacetoxyhydroborate (126 mg, 594 μmol) in DCE (10 mL) was stirred at rt for 3 hours, then the mixture was concentrated and the crude residue was purified by flash chromatography (eluting with 0% to 50% EtOAc in hexanes) to give compound 4a as yellow solid (50 mg). LCMS (M+H)⁺: 554.

### Step 2: Preparation of tert-butyl 4-(4-((((03R,5S)-1-(8-cyanoquinolin-5-yl)-5-methylpiperidin-3-yl)amino)methyl)-3-fluorophenyl)piperazine-1-carboxylate (Compound 4b)

A mixture of 5-((3R,5S)-3-((4-bromo-2-fluorobenzyl)amino)-5-methylpiperidin-1-yl)quinoline-8-carbonitrile (compound 4a, 50 mg, 110 μmol), tert-butyl piperazine-1-carboxylate (41 mg, 220 μmol, RuPhos G2 (8.57 mg, 11 μmol) and Cs₂CO₃ (108 mg, 331 μmol) in dioxane (5 mL) was charged with N₂, and the mixture was heated at 100° C. for 16 hours. After cooling, the solid was filtered off and the filtrate was concentrated. The residue was purified by preparative HPLC to give compound 4b as light yellow solid (30 mg). LCMS (M+H)⁺: 559.

### Step 3: Preparation of 5-[(3R,5S)-3-[(2-fluoro-4-piperazin-1-yl-phenyl)methylamino]-5-methyl-1-piperidyl]quinoline-8-carbonitrile (Example 4)

A mixture of tert-butyl 4-(4-((((3R,5 S)-1-(8-cyanoquinolin-5-yl)-5-methylpiperidin-3-yl)amino)methyl)-3-fluorophenyl)piperazine-1-carboxylate (compound 4b, 30 mg, 53.7 μmol) in 1M HCl in EtOAc (10 mL) was stirred at rt for 16 hours, then the solvent was removed in vacuo. The residue was lyophilized to give Example 4 as an orange solid (24 mg). LCMS(M+H)⁺: 459. ¹H NMR (400 MHz, METHANOL-d₄) δ ppm: 9.12 (dd, J=1.6, 4.6 Hz, 1H), 8.88 (dd, J=1.6, 8.6 Hz, 1H), 8.32 (d, J=8.2 Hz, 1H), 7.89 (dd, J=4.7, 8.6 Hz, 1H), 7.53 (t, J=8.9 Hz, 1H), 7.46 (d, J=8.2 Hz, 1H), 6.99-6.87 (m, 2H), 4.47-4.27 (m, 2H), 3.97 (br, 1H), 3.90-3.78 (m, 1H), 3.60-3.48 (m, 5H), 3.40-3.37 (m, 4H), 3.02 (t, J=11.1 Hz, 1H), 2.65 (t, J=11.7 Hz, 1H), 2.54 (br, 1H), 2.34-2.18 (m, 1H), 1.48-1.26 (m, 1H), 1.12 (d, J=6.6 Hz, 3H).

### Example 5

### 5-[(3S,5R)-3-methyl-5-[(5-piperazin-1-yl-2-pyridyl)methylamino]-1-piperidyl]quinoline-8-carbonitrile

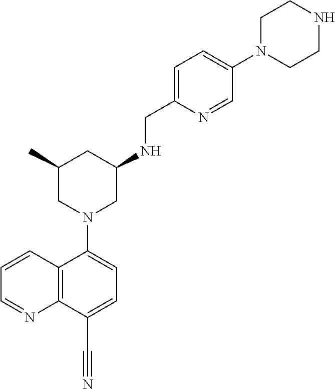 [SMILES: C[C@H]1C[C@@H](NCc2ccc(N3CCNCC3)cn2)CN(c2ccc(C#N)c3ncccc23)C1]

The title compound was prepared according to the following scheme:

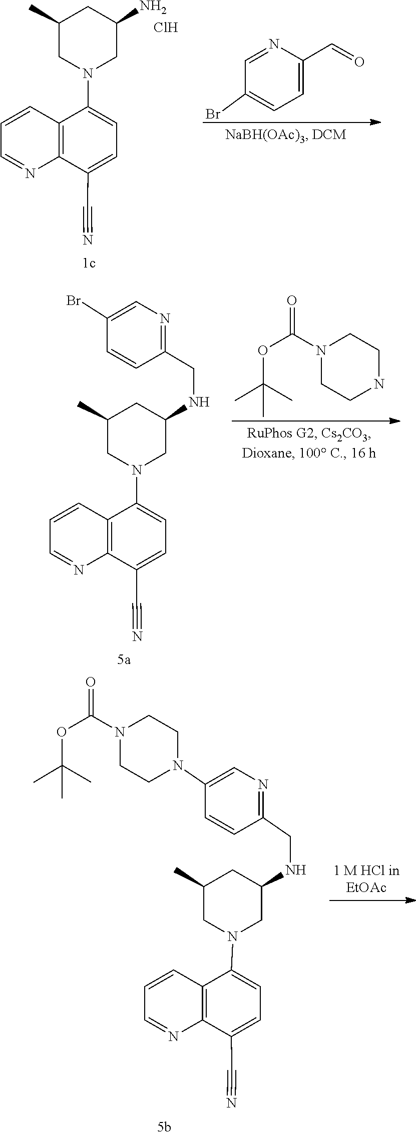

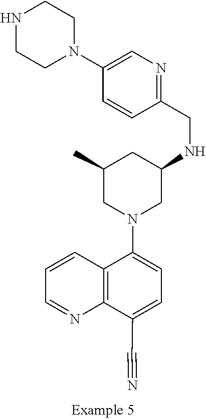 [SMILES: C[C@H]1C[C@@H](NCc2ccc(N3CCNCC3)cn2)CN(c2ccc(C#N)c3ncccc23)C1]

The title compound was prepared in analogy to the preparation of Example 4 by using 5-bromopyridine-2-carbaldehyde instead of 4-bromo-2-fluorobenzaldehyde. Example 5 was obtained as a light brown solid (14 mg). LCMS(M+H)⁺: 442, ¹H NMR (400 MHz, METHANOL-d₄) δ ppm: 9.08 (dd, J=1.6, 4.5 Hz, 1H), 8.77 (dd, J=1.6, 8.6 Hz, 1H), 8.51 (d, J=2.8 Hz, 1H), 8.27 (d, J=8.1 Hz, 1H), 7.87-7.66 (m, 3H), 7.42 (d, J=8.1 Hz, 1H), 4.63-4.45 (m, 2H), 3.98 (br, 1H), 3.87 (br, 1H), 3.70-3.60 (m, 4H), 3.58-3.47 (m, 1H), 3.45-3.38 (m, 4H), 3.04 (t, J=11.0 Hz, 1H), 2.63 (t, J=11.7 Hz, 1H), 2.53 (br, 1H), 2.27 (br, 1H), 1.43 (q, J=12.1 Hz, 1H), 1.12 (d, J=6.7 Hz, 3H).

### Example 6

### 5-[(3S,5R)-3-methyl-5-[[3-(4-methylpiperazin-1-yl)phenyl]methylamino]-1-piperidyl]quinoline-8-carbonitrile

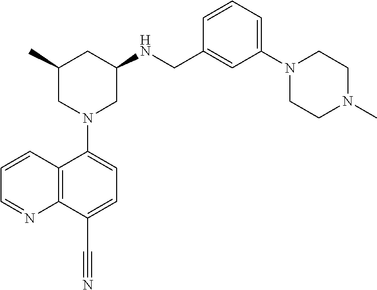 [SMILES: C[C@H]1C[C@@H](NCc2cccc(N3CCN(C)CC3)c2)CN(c2ccc(C#N)c3ncccc23)C1]

The title compound was prepared according to the following scheme:

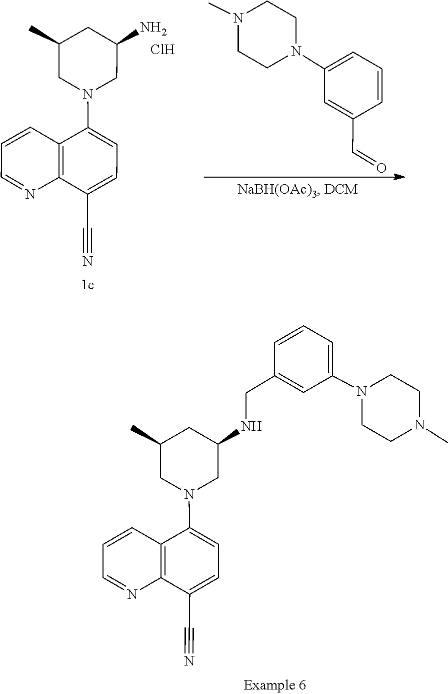 [SMILES: C[C@H]1C[C@@H](N)CN(c2ccc(C#N)c3ncccc23)C1 ; Cl ; CN1CCN(c2cccc(C=O)c2)CC1 ; C[C@H]1C[C@@H](NCc2cccc(N3CCN(C)CC3)c2)CN(c2ccc(C#N)c3ncccc23)C1]

A mixture of 5-((3R,5S)-3-amino-5-methylpiperidin-1-yl)quino line-8-carbonitrile (compound 1c, 50 mg, 188 μmol), 3-(4-methylpiperazin-1-yl)benzaldehyde (77 mg, 375 μmol, Et₃N (26.2 μL, 188 μmol), AcOH (10.7 μL, 188 μmol) and sodium triacetoxyborohydride (119 mg, 563 μmol) in DCM (10 mL) was stirred at 23° C. for 2 hours, then the solid was filtered. The filtrate was concentrated and purified by preparative HPLC give Example 6 as light yellow solid (3.6 mg). LCMS (M+H)⁺: 455, ¹H NMR (400 MHz, METHANOL-d₄) δ ppm: 8.86 (dd, J=1.7, 4.2 Hz, 1H), 8.31 (dd, J=1.6, 8.6 Hz, 1H), 8.00 (d, J=8.1 Hz, 1H), 7.49 (dd, J=4.3, 8.6 Hz, 1H), 7.21-7.04 (m, 2H), 6.90 (s, 1H), 6.85-6.72 (m, 2H), 3.72 (s, 2H), 3.59 (br d, J=9.0 Hz, 1H), 3.31 (br d, J=10.1 Hz, 1H), 3.15-3.05 (m, 4H), 2.97 (br t, J=10.8 Hz, 1H), 2.56-2.47 (m, 4H), 2.47-2.32 (m, 2H), 2.24 (s, 3H), 2.12 (br d, J=12.8 Hz, 1H), 1.95 (br s, 1H), 1.05-0.84 (m, 4H).

### Example 7

### 5-[(3S,5R)-3-methyl-5-[[4-(4-methylpiperazin-1-yl)phenyl]methylamino]-1-piperidyl]quinoline-8-carbonitrile

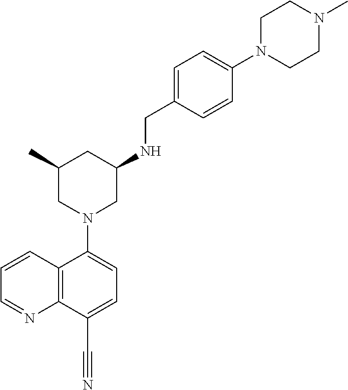 [SMILES: C[C@H]1C[C@@H](NCc2ccc(N3CCN(C)CC3)cc2)CN(c2ccc(C#N)c3ncccc23)C1]

The title compound was prepared in analogy to the preparation of Example 6 by using 4-(4-methylpiperazin-1-yl)benzaldehyde instead of 3-(4-methylpiperazin-1-yl)benzaldehyde. Example 7 was obtained as a light yellow solid (6 mg). LCMS(M+H)⁺: 455, ¹H NMR (400 MHz, METHANOL-d₄) δ ppm: 8.85 (dd, J=1.6, 4.3 Hz, 1H), 8.31 (dd, J=1.6, 8.6 Hz, 1H), 7.99 (d, J=8.1 Hz, 1H), 7.50 (dd, J=4.2, 8.6 Hz, 1H), 7.22-7.03 (m, 3H), 6.85 (d, J=8.7 Hz, 2H), 3.67 (s, 2H), 3.61-3.48 (m, 1H), 3.42-3.26 (m, 1H), 3.13-3.00 (m, 4H), 2.94 (tt, J=4.0, 10.9 Hz, 1H), 2.66-2.46 (m, 4H), 2.40 (dt, J=7.5, 11.2 Hz, 2H), 2.25 (s, 3H), 2.10 (br d, J=12.6 Hz, 1H), 2.01-1.85 (m, 1H), 1.09-0.78 (m, 4H).

### Example 8

### 5-[(3S,5R)-3-methyl-5-[(3-piperazin-1-ylphenyl)methylamino]-1-piperidyl]quinoline-8-carbonitrile

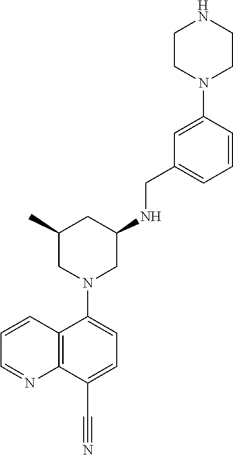 [SMILES: C[C@H]1C[C@@H](NCc2cccc(N3CCNCC3)c2)CN(c2ccc(C#N)c3ncccc23)C1]

The title compound was prepared in analogy to the preparation of Example 1 by using tert-butyl 4-(3-formylphenyl)piperazine-1-carboxylate instead of compound 1d. Example 8 was obtained as a light yellow solid (53 mg). LCMS(M+H)⁺: 441, ¹H NMR (400 MHz, METHANOL-d₄) δ ppm: 9.12 (dd, J=1.5, 4.7 Hz, 1H), 8.88 (dd, J=1.5, 8.6 Hz, 1H), 8.32 (d, J=8.2 Hz, 1H), 7.89 (dd, J=4.7, 8.6 Hz, 1H), 7.52-7.35 (m, 3H), 7.17-7.06 (m, 2H), 4.43-4.27 (m, 2H), 4.01 (br d, J=9.8 Hz, 1H), 3.85 (br t, J=11.4 Hz, 1H), 3.62-3.48 (m, 5H), 3.45-3.38 (m, 4H), 3.06 (t, J=11.1 Hz, 1H), 2.66 (t, J=11.7 Hz, 1H), 2.55 (br d, J=11.9 Hz, 1H), 2.35-2.17 (m, 1H), 1.44 (q, J=12.0 Hz, 1H), 1.12 (d, J=6.6 Hz, 3H).

### Example 9

### 5-[(3S,5R)-3-methyl-5-[methyl-[(4-piperazin-1-ylphenyl)methyl]amino]-1-piperidyl]quinoline-8-carbonitrile

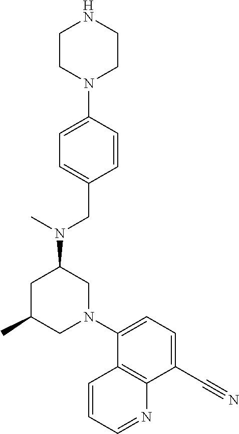 [SMILES: C[C@H]1C[C@@H](N(C)Cc2ccc(N3CCNCC3)cc2)CN(c2ccc(C#N)c3ncccc23)C1]

The title compound was prepared according to the following scheme:

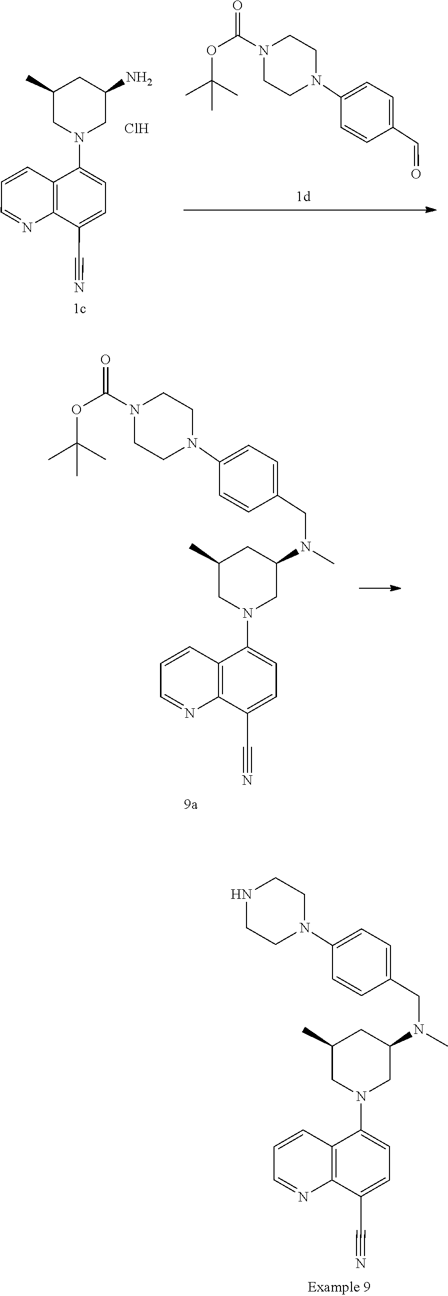 [SMILES: C[C@H]1C[C@@H](N)CN(c2ccc(C#N)c3ncccc23)C1 ; Cl ; CC(C)(C)OC(=O)N1CCN(c2ccc(C=O)cc2)CC1 ; C[C@H]1C[C@@H](N(C)Cc2ccc(N3CCN(C(=O)OC(C)(C)C)CC3)cc2)CN(c2ccc(C#N)c3ncccc23)C1 ; C[C@H]1C[C@@H](N(C)Cc2ccc(N3CCNCC3)cc2)CN(c2ccc(C#N)c3ncccc23)C1]

### Step 1: Preparation of tert-butyl 4-[4-[[[(3R,5S)-1-(8-cyano-5-quinolyl)-5-methyl-3-piperidyl]-methyl-amino]methyl]phenyl]piperazine-1-carboxylate (Compound 9a)

A mixture of 5-((3R,5S)-3-amino-5-methylpiperidin-1-yl)quinoline-8-carbonitrile hydrochloride (compound 1c, 50 mg, 165 μmol), tert-butyl 4-(4-formylphenyl)piperazine-1-carboxylate (52.7 mg, 182 μmol), TEA (23 μL, 165 μmol), AcOH (9.45 μL, 165 μmol) and sodium triacetoxyborohydride (105 mg, 495 μmol, Eq: 3) in DCM (10 mL) was stirred at rt for 2 hours, then formaldehyde (4.96 mg, 165 μmol, Eq: 1) was added and stirred at rt for 2 hours. The solid was filtered, and the filtrate was concentrated and purified by silica gel (0%-70% PE in EA, EA contains 10% MeOH) to give compound 9a as a light yellow solid (40 mg). LCMS(M+H)⁺: 555.

### Step 2: Preparation of 5-[(3S,5R)-3-methyl-5-[methyl-[(4-piperazin-1-ylphenyl)methyl]amino]-1-piperidyl]quinoline-8-carbonitrile (Example 9)

A mixture of tert-butyl 4-(4-((((3R,5S)-1-(8-cyanoquinolin-5-yl)-5-methylpiperidin-3-yl)(methyl)amino)methyl)phenyl)piperazine-1-carboxylate (compound 9a, 40 mg, 72.1 μmol) in 1M HCl in EA (10 mL) was stirred at rt for 16 hours, then the mixture was filtered to give Example 9 as a light yellow solid (26 mg). LCMS(M+H)⁺: 455, ¹H NMR (400 MHz, METHANOL-d₄) δ ppm: 9.08-8.98 (m, 1H), 8.60 (ddd, J=1.5, 8.6, 11.9 Hz, 1H), 8.20 (dd, J=3.2, 7.9 Hz, 1H), 7.71 (ddd, J=4.2, 8.6, 10.9 Hz, 1H), 7.52 (d, J=8.7 Hz, 1H), 7.48-7.33 (m, 2H), 7.13 (dd, J=8.7, 17.9 Hz, 2H), 4.58 (dd, J=5.4, 13.1 Hz, 1H), 4.27 (dd, J=4.6, 13.1 Hz, 1H), 3.99-3.82 (m, 2H), 3.58-3.44 (m, 5H), 3.45-3.37 (m, 4H), 3.22 (br d, J=8.1 Hz, 1H), 2.84 (d, J=6.5 Hz, 3H), 2.61 (dt, J=6.7, 11.7 Hz, 1H), 2.46 (br s, 1H), 2.33-2.19 (m, 1H), 1.71-1.52 (m, 1H), 1.17-1.13 (m, 3H).

### Example 10

### 5-[(3S,5R)-3-methyl-5-[methyl-[(4-piperazin-1-ylphenyl)methyl]amino]-1-piperidyl]quinoline-8-carbonitrile

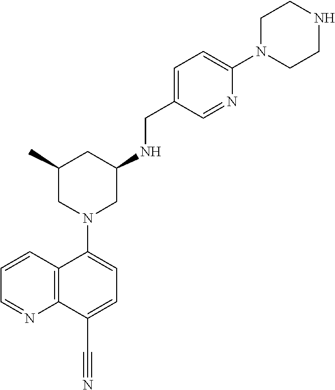 [SMILES: C[C@H]1C[C@@H](NCc2ccc(N3CCNCC3)nc2)CN(c2ccc(C#N)c3ncccc23)C1]

The title compound was prepared in analogy to the preparation of Example 4 by using 5-bromopyridine-2-carbaldehyde instead of 4-bromo-2-fluorobenzaldehyde. Example 10 was obtained as a light brown solid (28 mg). LCMS(M+H)⁺: 442, ¹H NMR (400 MHz, METHANOL-d₄) δ ppm: 9.10 (br s, 1H), 8.88 (br s, 1H), 8.49-8.22 (m, 3H), 7.87 (br s, 1H), 7.60-7.39 (m, 2H), 4.45 (br s, 2H), 4.19-3.80 (m, 6H), 3.64-3.42 (m, 5H), 3.18-3.03 (m, 1H), 2.78-2.50 (m, 2H), 2.30 (br s, 1H), 1.48 (br s, 1H), 1.13 (br s, 3H).

### Example 11

### 5-[(3S,5R)-3-methyl-5-(1,2,3,4-tetrahydroisoquinolin-6-ylmethylamino)-1-piperidyl]quinoline-8-carbonitrile

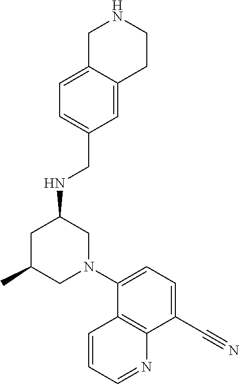 [SMILES: C[C@H]1C[C@@H](NCc2ccc3c(c2)CCNC3)CN(c2ccc(C#N)c3ncccc23)C1]

The title compound was prepared in analogy to the preparation of Example 2 by using tert-butyl 6-bromo-3,4-dihydro-1H-isoquinoline-2-carboxylate instead of compound 2b. Example 11 was obtained as a light brown solid (35 mg). LCMS(M+H)⁺: 412, ¹H NMR (400 MHz, METHANOL-d₄) δ ppm: 8.98 (dd, J=1.5, 4.6 Hz, 1H), 8.73 (dd, J=1.5, 8.6 Hz, 1H), 8.18 (d, J=8.1 Hz, 1H), 7.75 (dd, J=4.6, 8.6 Hz, 1H), 7.46-7.37 (m, 2H), 7.33 (d, J=8.1 Hz, 1H), 7.24 (d, J=7.8 Hz, 1H), 4.36-4.19 (m, 4H), 3.88 (br d, J=9.8 Hz, 1H), 3.80-3.66 (m, 1H), 3.43 (t, J=6.4 Hz, 3H), 3.08 (t, J=6.4 Hz, 2H), 2.93 (t, J=11.1 Hz, 1H), 2.52 (t, J=11.7 Hz, 1H), 2.44 (br d, J=12.1 Hz, 1H), 2.23-2.08 (m, 1H), 1.38-1.25 (m, 1H), 1.01 (d, J=6.6 Hz, 3H).

### Example 12

### 5-[(3S,5R)-3-methyl-5-(5-piperazin-1-ylisoindolin-2-yl)-1-piperidyl]quinoline-8-carbonitrile

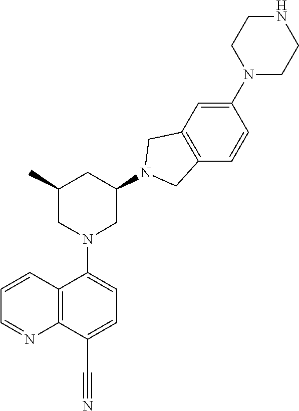 [SMILES: C[C@H]1C[C@@H](N2Cc3ccc(N4CCNCC4)cc3C2)CN(c2ccc(C#N)c3ncccc23)C1]

The title compound was prepared according to the following scheme:

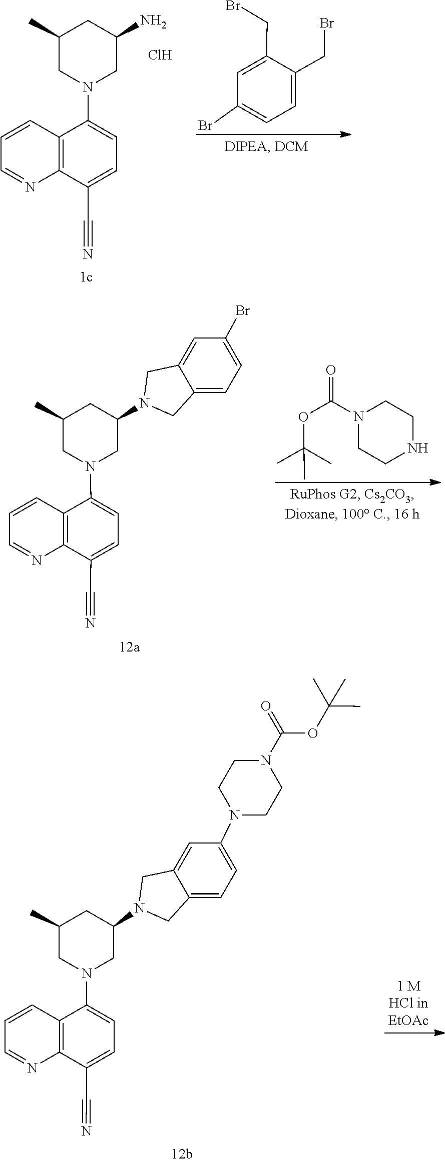 [SMILES: C[C@H]1C[C@@H](N)CN(c2ccc(C#N)c3ncccc23)C1 ; Cl ; BrCc1ccc(Br)cc1CBr ; C[C@H]1C[C@@H](N2Cc3ccc(Br)cc3C2)CN(c2ccc(C#N)c3ncccc23)C1 ; CC(C)(C)OC(=O)N1CCNCC1 ; C[C@H]1C[C@@H](N2Cc3ccc(N4CCN(C(=O)OC(C)(C)C)CC4)cc3C2)CN(c2ccc(C#N)c3ncccc23)C1]

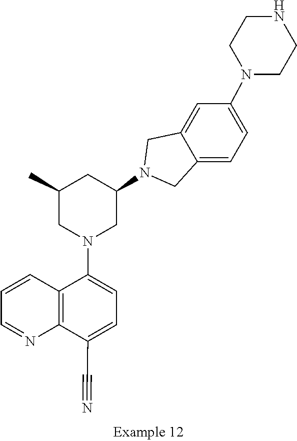 [SMILES: C[C@H]1C[C@@H](N2Cc3ccc(N4CCNCC4)cc3C2)CN(c2ccc(C#N)c3ncccc23)C1]

### Step 1: Preparation of 5-((3R,5S)-3-(5-bromoisoindolin-2-yl)-5-methylpiperidin-1-yl)quinoline-8-carbonitrile (Compound 12a)

A mixture of 5-((3R,5 S)-3-amino-5-methylpiperidin-1-yl)quino line-8-carbonitrile hydrochloride (compound 1c, 50 mg, 165 μmol), 4-bromo-1,2-bis(bromomethyl)benzene (170 mg, 495 μmol) and DIPEA (64 mg, 86.5 μL, 495 μmol in DCM (10 mL) was stirred at 40° C. for 16 hours, then the mixture was concentrated and the crude residue was purified by flash chromatography (eluting with 0% to 60% EtOAc in hexanes) to give compound 12a as a light yellow solid (50 mg). LCMS(M+H)⁺: 448.

### Step 2: Preparation of tert-butyl 4-(2-((3R,5S)-1-(8-cyanoquinolin-5-yl)-5-methylpiperidin-3-yl)isoindolin-5-yl)piperazine-1-carboxylate (Compound 12b)

A mixture of 5-((3R,5S)-3-(5-bromoisoindolin-2-yl)-5-methylpiperidin-1-yl)quinoline-8-carbonitrile (compound 12a, 50 mg, 112 μmol, tert-butyl piperazine-1-carboxylate (41.6 mg, 224 μmol, RuPhos G2 (8.68 mg, 11.2 μmol) and Cs₂CO₃ (72.8 mg, 224 μmol in dioxane (5 mL) was charged with N₂. The mixture was heated at 100° C. for 16 hours. After cooling, the undissolved material was filtered off and washed with EA (10 mL). The combined filtrates were concentrated and purified by flash chromatography (eluting with 10% to 80% EtOAc in hexanes) to give compound 12b as a light yellow solid (30 mg). LCMS(M+H)⁺: 553.

### Step 3: Preparation of 5-[(3S,5R)-3-methyl-5-(5-piperazin-1-ylisoindolin-2-yl)-1-piperidyl]quinoline-8-carbonitrile (Example 12)

A mixture of tert-butyl 4-(2-43R,5S)-1-(8-cyanoquinolin-5-yl)-5-methylpiperidin-3-yl)isoindolin-5-yl)piperazine-1-carboxylate (compound 12b, 30 mg, 54.3 μmol) in 1 M HCl in EA (10 mL) was stirred at rt for 16 hour, then the reaction mixture was concentrated and the crude residue was purified by preparative HPLC to give Example 12 as a light yellow solid (4 mg). LCMS(M+H)⁺: 453, ¹H NMR (400 MHz, METHANOL-d₄) δ ppm: 8.88 (dd, J=1.6, 4.2 Hz, 1H), 8.50 (dd, J=1.6, 8.6 Hz, 1H), 8.05 (d, J=8.1 Hz, 1H), 7.56 (dd, J=4.2, 8.6 Hz, 1H), 7.20 (d, J=8.1 Hz, 1H), 7.04 (d, J=8.1 Hz, 1H), 6.89-6.70 (m, 2H), 3.91 (br d, J=11.4 Hz, 4H), 3.71 (br d, J=11.2 Hz, 1H), 3.45-3.31 (m, 1H), 3.08-2.96 (m, 5H), 2.94-2.85 (m, 4H), 2.62 (t, J=10.9 Hz, 1H), 2.43 (t, J=11.4 Hz, 1H), 2.26 (br d, J=12.5 Hz, 1H), 2.08 (br s, 1H), 1.16-1.02 (m, 1H), 0.97 (d, J=6.7 Hz, 3H).

### Example 13

### 5-[(3R,5S)-3-[[4-(3-aminopyrrolidin-1-yl)phenyl]methylamino]-5-methyl-1-piperidyl]quinoline-8-carbonitrile

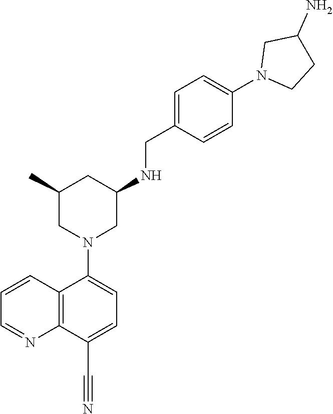 [SMILES: C[C@H]1C[C@@H](NCc2ccc(N3CCC(N)C3)cc2)CN(c2ccc(C#N)c3ncccc23)C1]

The title compound was prepared according to the following scheme:

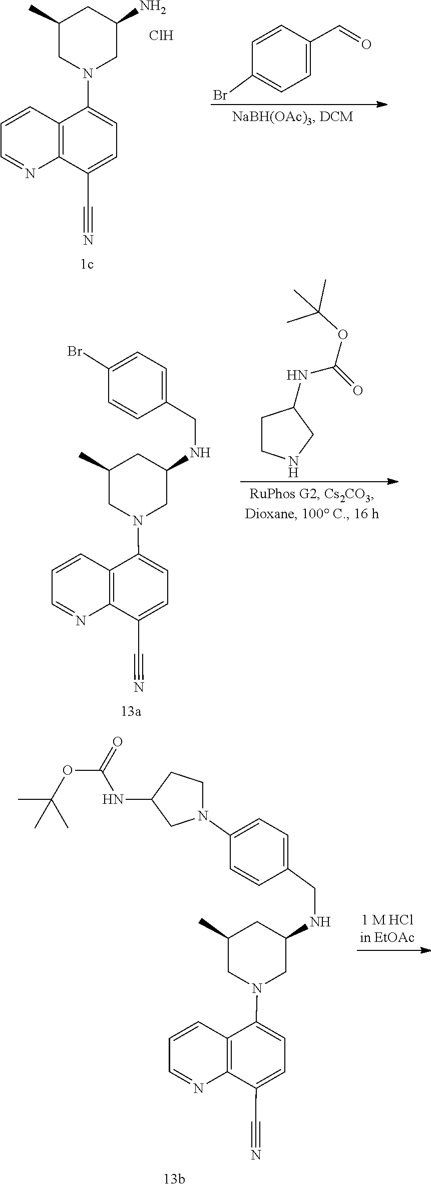 [SMILES: C[C@H]1C[C@@H](N)CN(c2ccc(C#N)c3ncccc23)C1 ; Cl ; O=Cc1ccc(Br)cc1 ; C[C@H]1C[C@@H](NCc2ccc(Br)cc2)CN(c2ccc(C#N)c3ncccc23)C1 ; CC(C)(C)OC(=O)NC1CCNC1 ; C[C@H]1C[C@@H](NCc2ccc(N3CCC(NC(=O)OC(C)(C)C)C3)cc2)CN(c2ccc(C#N)c3ncccc23)C1]

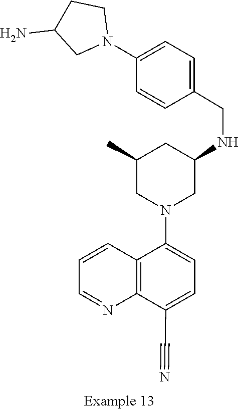 [SMILES: C[C@H]1C[C@@H](NCc2ccc(N3CCC(N)C3)cc2)CN(c2ccc(C#N)c3ncccc23)C1]

The title compound was prepared in analogy to the preparation of Example 4 by using 5-bromopyridine-2-carbaldehyde instead of 4-bromo-2-fluorobenzaldehyde and tert-butyl N-pyrrolidin-3-ylcarbamate instead of tert-butyl piperazine-1-carboxylate. Example 13 was obtained as a light brown solid (2 mg). LCMS(M+H)⁺: 441, ¹H NMR (400 MHz, METHANOL-d₄) δ ppm: 8.98 (dd, J=1.5, 4.2 Hz, 1H), 8.46 (d, J=8.2 Hz, 1H), 8.13 (d, J=7.9 Hz, 1H), 7.65 (dd, J=4.1, 8.4 Hz, 1H), 7.24-7.19 (m, 3H), 6.59 (d, J=8.4 Hz, 2H), 3.85-3.67 (m, 4H), 3.58-3.39 (m, 4H), 3.21-3.10 (m, 2H), 2.62-2.49 (m, 2H), 2.41-2.19 (m, 2H), 2.08 (br s, 1H), 2.01-1.91 (m, 1H), 1.14-0.99 (m, 4H).

### Example 14

### 5-[(3R,5S)-3-[[4-(3-amino-1-piperidyl)phenyl]methylamino]-5-methyl-1-piperidyl]quinoline-8-carbonitrile

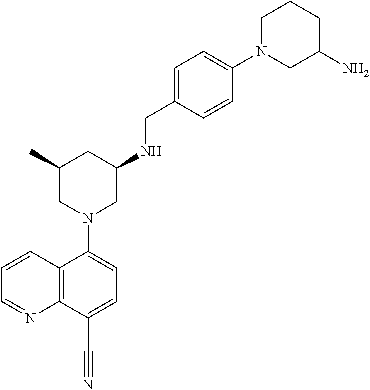 [SMILES: C[C@H]1C[C@@H](NCc2ccc(N3CCCC(N)C3)cc2)CN(c2ccc(C#N)c3ncccc23)C1]

The title compound was prepared according to the following scheme:

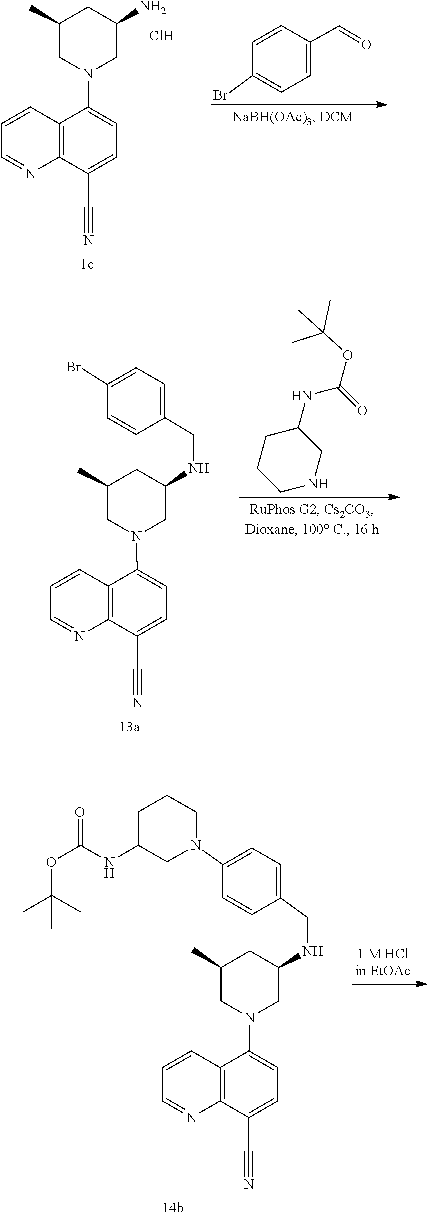 [SMILES: C[C@H]1C[C@@H](N)CN(c2ccc(C#N)c3ncccc23)C1 ; Cl ; O=Cc1ccc(Br)cc1 ; C[C@H]1C[C@@H](NCc2ccc(Br)cc2)CN(c2ccc(C#N)c3ncccc23)C1 ; CC(C)(C)OC(=O)NC1CCCNC1 ; C[C@H]1C[C@@H](NCc2ccc(N3CCCC(NC(=O)OC(C)(C)C)C3)cc2)CN(c2ccc(C#N)c3ncccc23)C1]

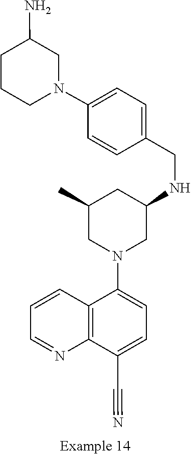 [SMILES: C[C@H]1C[C@@H](NCc2ccc(N3CCCC(N)C3)cc2)CN(c2ccc(C#N)c3ncccc23)C1]

The title compound was prepared in analogy to the preparation of Example 4 by using 5-bromopyridine-2-carbaldehyde instead of 4-bromo-2-fluorobenzaldehyde and tert-butyl N-(3-piperidyl)carbamate instead of tert-butyl piperazine-1-carboxylate. Example 14 was obtained as a light brown solid (2 mg). LCMS(M+H)⁺: 455, ¹H NMR (400 MHz, METHANOL-d₄) δ ppm 9.03 (d, J=4.2 Hz, 1H), 8.43 (d, J=8.6 Hz, 1H), 8.24-8.11 (m, 1H), 7.75-7.60 (m, 1H), 7.33-7.29 (m, 3H), 7.08 (br d, J=8.6 Hz, 2H), 3.83 (br d, J=10.6 Hz, 1H), 3.62 (br d, J=11.9 Hz, 2H), 3.54-3.42 (m, 4H), 3.21-3.05 (m, 4H), 2.68-2.38 (m, 2H), 2.11-2.04 (m, 2H), 1.97-1.92 (m, 2H), 1.85-1.69 (m, 2H), 1.12 (d, J=6.5 Hz, 3H).

### Example 15

### 5-[(3R,5S)-3-[[4-(2,6-dimethyl-4-pyridyl)phenyl]methylamino]-5-methyl-1-piperidyl]quinoline-8-carbonitrile

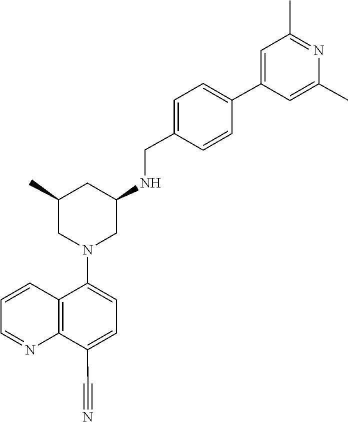 [SMILES: Cc1cc(-c2ccc(CN[C@@H]3C[C@H](C)CN(c4ccc(C#N)c5ncccc45)C3)cc2)cc(C)n1]

The title compound was prepared according to the following scheme:

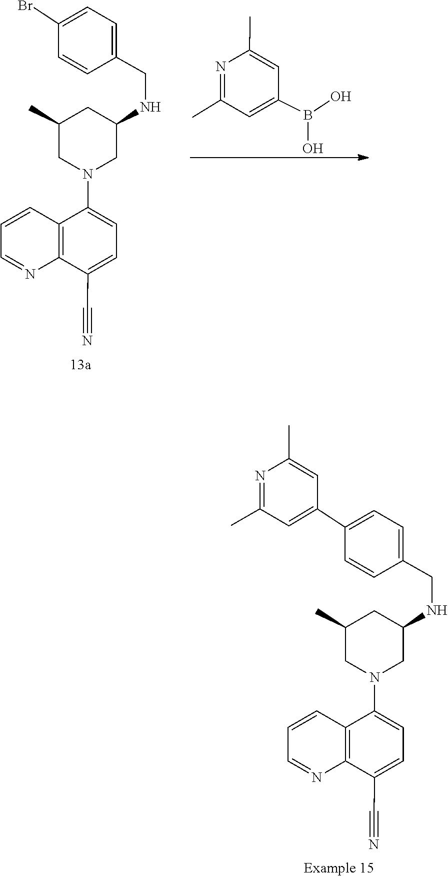 [SMILES: C[C@H]1C[C@@H](NCc2ccc(Br)cc2)CN(c2ccc(C#N)c3ncccc23)C1 ; Cc1cc(B(O)O)cc(C)n1 ; Cc1cc(-c2ccc(CN[C@@H]3C[C@H](C)CN(c4ccc(C#N)c5ncccc45)C3)cc2)cc(C)n1]

A mixture of 5-((3R,5S)-3-((4-bromobenzyl)amino)-5-methylpiperidin-1-yl)quinoline-8-carbonitrile (compound 13a, 50 mg, 115 μmol), (2,6-dimethylpyridin-4-yl)boronic acid (34.7 mg, 230 μmol), Na₂CO₃ (24.3 mg, 230 μmol) and PdCl₂(DPPF) (9.38 mg, 11.5 μmol) in dioxane/water (5:1) (6 mL) was charged with N₂, and the mixture was heated at 100° C. for 16 hours. After cooling, the solid was filtered off and washed with EA (10 mL). The combined filtrates were concentrated, and the residue was purified by preparative HPLC to give Example 15 as a light yellow solid (40 mg). LCMS(M+H)⁺: 462, ¹H NMR (400 MHz, METHANOL-d₄) δ ppm: 9.03 (dd, J=1.7, 4.2 Hz, 1H), 8.60 (dd, J=1.6, 8.6 Hz, 1H), 8.20 (d, J=8.1 Hz, 1H), 8.11-7.99 (m, 4H), 7.78 (d, J=8.3 Hz, 2H), 7.70 (dd, J=4.3, 8.6 Hz, 1H), 7.35 (d, J=7.9 Hz, 1H), 4.49 (q, J=13.0 Hz, 2H), 3.97-3.76 (m, 2H), 3.58-3.45 (m, 1H), 2.94 (t, J=10.9 Hz, 1H), 2.81 (s, 6H), 2.63-2.49 (m, 2H), 2.28 (br s, 1H), 1.37 (q, J=11.9 Hz, 114), 1.13 (d, J=6.6 Hz, 3H).

### Example 16

### 5-[(3S,5R)-3-methyl-5-[[2-(4-methylpiperazin-1-yl)pyrimidin-5-yl]methylamino]-1-piperidyl]quinoline-8-carbonitrile

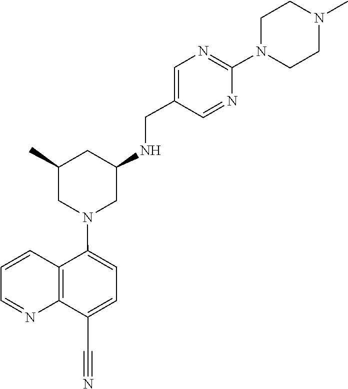 [SMILES: C[C@H]1C[C@@H](NCc2cnc(N3CCN(C)CC3)nc2)CN(c2ccc(C#N)c3ncccc23)C1]

The title compound was prepared according to the following scheme:

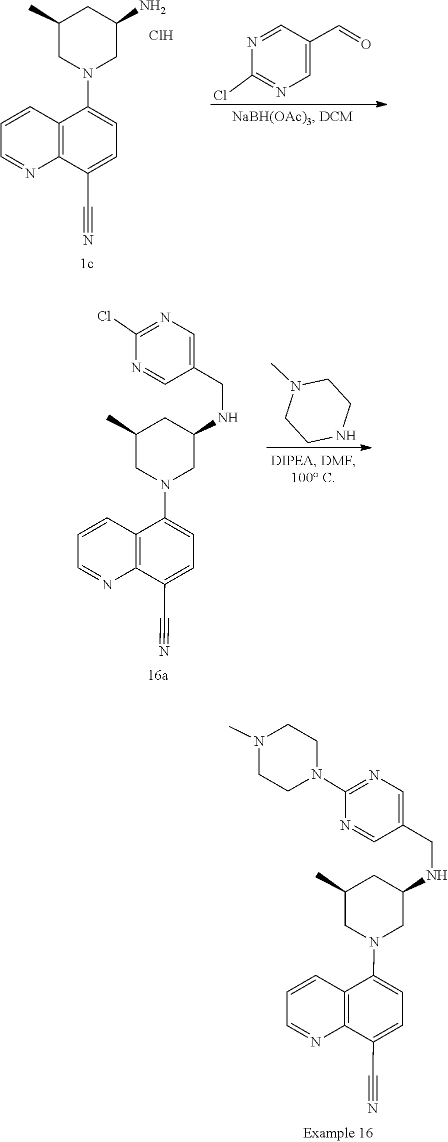

### Step 1: Preparation of 5-((3R,5S)-3-(((2-chloropyrimidin-5-yl)methyl)amino)-5-methylpiperidin-1-yl)quinoline-8-carbonitrile (Compound 16a)

A mixture of 5-((3R,5S)-3-amino-5-methylpiperidin-1-yl)quinoline-8-carbonitrile hydrochloride (compound 1c, 100 mg, 330 μmol), 2-chloropyrimidine-5-carbaldehyde (47.1 mg, 330 μmol), DIPEA (57.7 μL, 330 μmol), AcOH (18.9 μL, 330 μmol) and sodium triacetoxyborohydride (210 mg, 991 μmol) in DCM (15 mL) was stirred at rt for 2 hours, then the reaction mixture was diluted with EtOAc, washed with water and brine. The organic layer was concentrated and the residue was purified by silica gel column to give compound 16a as a light yellow oil (100 mg). LCMS(M+H)⁺: 393.

### Step 2: Preparation of 5-[(3S,5R)-3-methyl-5-[[2-(4-methylpiperazin-1-yl)pyrimidin-5-yl]methylamino]-1-piperidyl]quinoline-8-carbonitrile (Example 16)

A mixture of 5-(3R,5S)-3-(((2-chloropyrimidin-5-yl)methyl)amino)-5-methylpiperidin-1-yl)quinoline-8-carbonitrile (compound 16a, 50 mg, 127 μmol), 1-methylpiperazine (127 mg, 1.27 mmol) and DIPEA (22 μL, 1.27 mmol) in DMF (5 mL) was heated at 100° C. for 16 hours. The reaction solution was purified by preparative HPLC to give Example 16 as a light yellow solid (40 mg). LCMS(M+H)⁺: 457, ¹H NMR (400 MHz, METHANOL-d₄) δ ppm: 9.03 (dd, J=1.6, 4.2 Hz, 1H), 8.68-8.52 (m, 3H), 8.20 (d, J=8.1 Hz, 1H), 7.70 (dd, J=4.3, 8.6 Hz, 1H), 7.36 (d, J=8.1 Hz, 1H), 4.38-4.21 (m, 2H), 3.90 (br d, J=10.9 Hz, 1H), 3.85-3.69 (m, 2H), 3.60 (br s, 1H), 3.54-3.47 (m, 1H), 3.37-3.33 (m, 4H) 3.28-3.22 (m, 1H), 2.98 (s, 3H), 2.94-2.87 (m, 1H), 2.65-2.46 (m, 3H), 2.27 (br s, 1H), 1.37-1.28 (m, 1H), 1.12 (d, J=6.6 Hz, 3H).

### Example 17

### 5-[(3S,5R)-3-methyl-5-[(2-piperazin-1-ylpyrimidin-5-yl)methylamino]-1-piperidyl]quinoline-8-carbonitrile

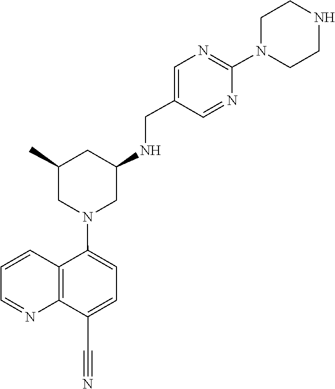 [SMILES: C[C@H]1C[C@@H](NCc2cnc(N3CCNCC3)nc2)CN(c2ccc(C#N)c3ncccc23)C1]

The title compound was prepared according to the following scheme:

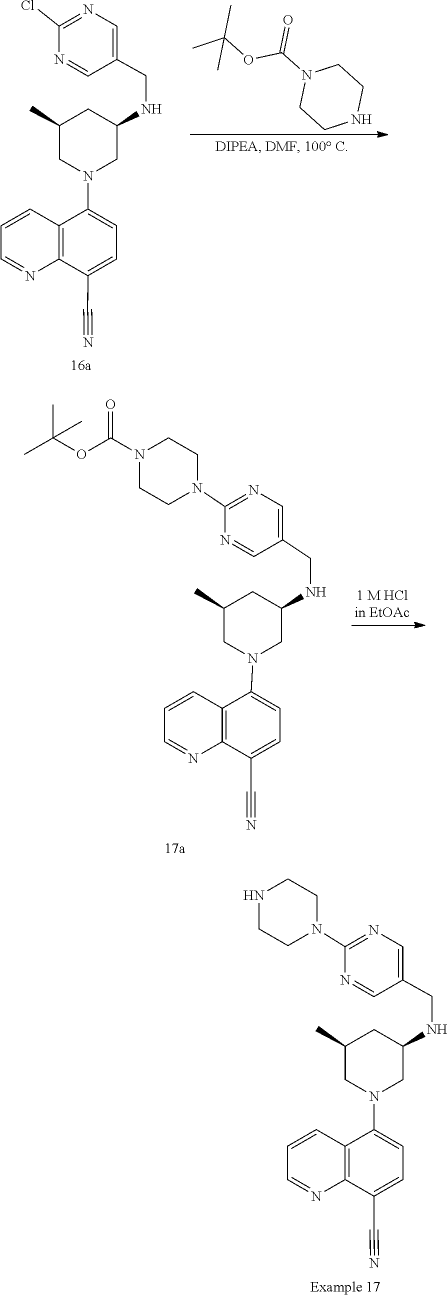 [SMILES: C[C@H]1C[C@@H](NCc2cnc(Cl)nc2)CN(c2ccc(C#N)c3ncccc23)C1 ; CC(C)(C)OC(=O)N1CCNCC1 ; C[C@H]1C[C@@H](NCc2cnc(N3CCN(C(=O)OC(C)(C)C)CC3)nc2)CN(c2ccc(C#N)c3ncccc23)C1 ; C[C@H]1C[C@@H](NCc2cnc(N3CCNCC3)nc2)CN(c2ccc(C#N)c3ncccc23)C1]

### Step 1: Preparation of tert-butyl 4-(5-((((3R,5S)-1-(8-cyanoquinolin-5-yl)-5-methylpiperidin-3-yl)amino)methyl)pyrimidin-2-yl)piperazine-1-carboxylate (Compound 17a)

A mixture of 5-(3R,5S)-3-(((2-chloropyrimidin-5-yl)methyl)amino)-5-methylpiperidin-1-yl)quinoline-8-carbonitrile (compound 16a, 50 mg, 127 μmol), tert-butyl piperazine-1-carboxylate (237 mg, 1.27 mmol) and DIPEA (222 μL, 1.27 mmol) in DMF (5 mL) was heated at 100° C. for 16 hours. The reaction solution was purified by preparative HPLC to give compound 17a as a light yellow solid (30 mg). LCMS(M+H)⁺: 543.

### Step 2: Preparation of 5-[(3S,5R)-3-methyl-5-[(2-piperazin-1-ylpyrimidin-5-yl)methylamino]-1-piperidyl]quinoline-8-carbonitrile (Example 17)

A mixture of tert-butyl 4-(5-((((3R,5S)-1-(8-cyanoquinolin-5-yl)-5-methylpiperidin-3-yl)amino)methyl)pyrimidin-2-yl)piperazine-1-carboxylate (compound 17a, 30 mg, 55.3 μmol) in 1 M HCl in EA (5 mL) was stirred at rt for 16 hours, then the reaction was concentrated to give Example 17 as an orange solid (20 mg). LCMS(M+H)⁺: 443, ¹H NMR (400 MHz, METHANOL-d₄) δ ppm: 9.10 (dd, J=1.5, 4.5 Hz, 1H), 8.84 (dd, J=1.5, 8.6 Hz, 1H), 8.63 (s, 2H), 8.30 (d, J=8.1 Hz, 1H), 7.86 (dd, J=4.6, 8.6 Hz, 1H), 7.45 (d, J=8.1 Hz, 1H), 4.42-4.23 (m, 2H), 4.20-4.11 (m, 4H), 3.99 (br d, J=10.8 Hz, 1H), 3.85 (br t, J=11.2 Hz, 1H), 3.59-3.48 (m, 1H), 3.33-3.25 (m, 4H), 3.01 (t, J=11.1 Hz, 1H), 2.64 (t, J=11.7 Hz, 1H), 2.55 (br d, J=12.5 Hz, 1H), 2.26 (br s, 1H), 1.40 (q, J=12.0 Hz, 1H), 1.13 (d, J=6.6 Hz, 3H).

### Example 18

### 7-[(3S,5R)-3-methyl-5-[[5-(4-methylpiperazin-1-yl)-2-pyridyl]methylamino]-1-piperidyl]-1,3-benzothiazole-4-carbonitrile

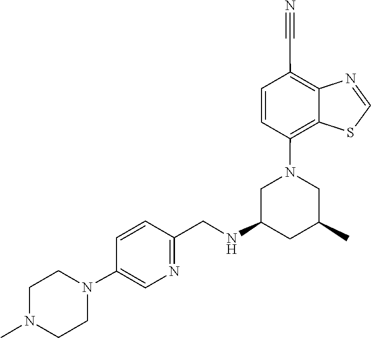 [SMILES: C[C@H]1C[C@@H](NCc2ccc(N3CCN(C)CC3)cn2)CN(c2ccc(C#N)c3ncsc23)C1]

The title compound was prepared according to the following scheme:

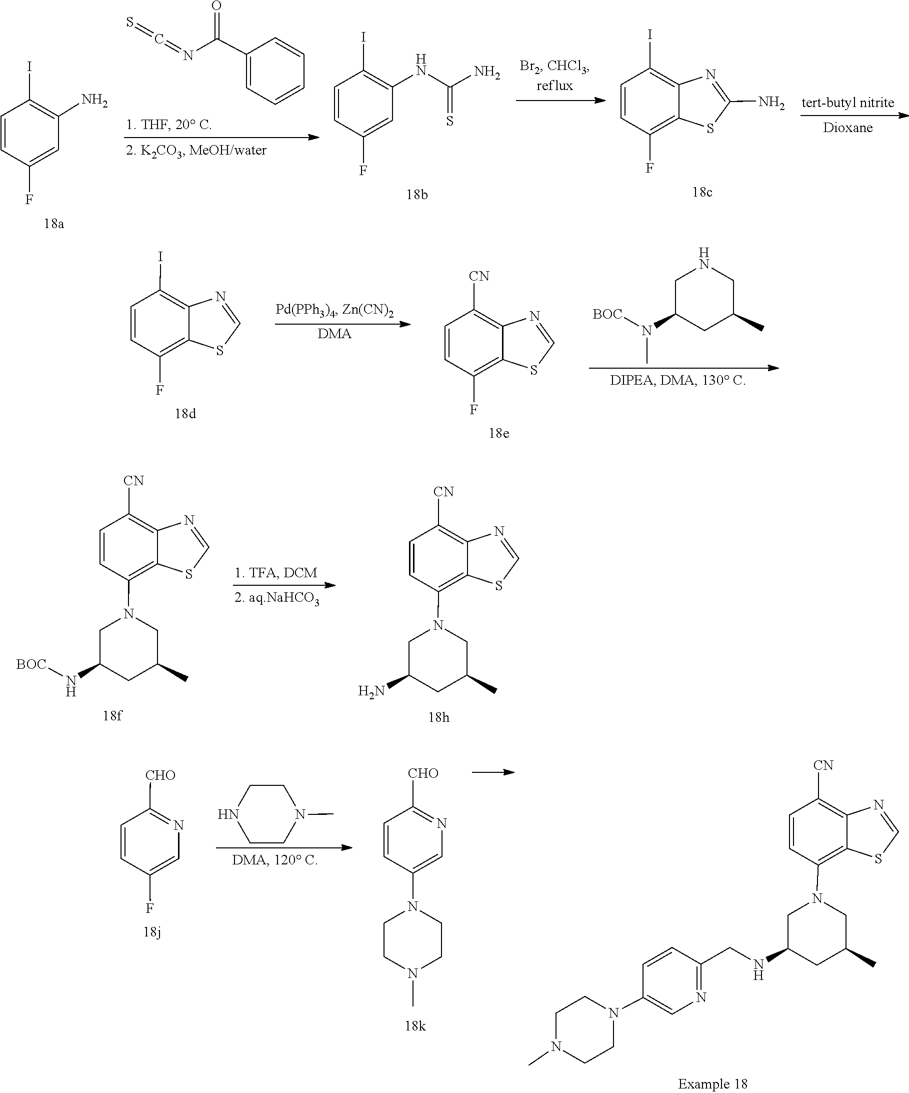

### Step 1: Preparation of (5-fluoro-2-iodo-phenyl)thiourea (Compound 18b)

To a solution of 5-fluoro-2-iodo-aniline (compound 18a, 20.0 g, 84.38 mmol) in THF (375 mL) was added benzoyl isothiocyanate (27.5 g, 168.8 mmol) at 10-20° C. The reaction was stirred at 20° C. for 16 hours. The solvent was removed and the solid was washed with EtOH (80 mL). The intermediate was dissolved in methanol (429 mL), and then potassium carbonate (35 g, 253 mmol) in water (50 mL) was added. The reaction was heated at 70° C. for 4 hours. The solvent was removed and the residue was purified by silica gel to give compound 18b (19 g) as a white solid.

### Step 2: Preparation of 7-fluoro-4-iodo-1,3-benzothiazol-2-amine (Compound 18c)

To a solution of (5-fluoro-2-iodo-phenyl)thiourea (compound 18b, 20 g, 67.54 mmol) in chloroform (359 mL) was added bromine (3.47 mL, 67.54 mmol) at 10° C. The reaction was heated at 80° C. for 4 hours. After cooled to rt, MeOH (50 mL) was added and the reaction mixture was quenched with aq. Na₂S₂O₃ and aq. NaHCO₃. After separation, the organic layer was washed with brine (100 mL), and dried over anhydrous Na₂SO₄. After filtration, the solvent was removed and the residue was purified by silica gel to give compound 18c (67 g) as a white solid. LCMS(M+H)⁺: 295.

### Step 3: Preparation of 7-fluoro-4-iodo-1,3-benzothiazole (Compound 18d)

To a solution of 7-fluoro-4-iodo-1,3-benzothiazol-2-amine (compound 18c, 4 g, 13.6 mmol) in 1,4-dioxane (84.26 mL) was added tert-butyl nitrite (2.8 g, 27.2 mmol). The reaction was heated at 80° C. for 18 hours. The solvent was removed and the residue was purified by silica gel to give compound 18d (35 g) as a yellow solid. LCMS(M+H)⁺: 280.

### Step 4: Preparation of 7-fluoro-1,3-benzothiazole-4-carbonitrile (Compound 18e)

Zinc cyanide (1.5 mL, 23.65 mmol) and tetrakis(triphenylphosphine)palladium(0) (660.0 mg, 0.570 mmol) was added to a solution of 7-fluoro-4-iodo-1,3-benzothiazole (compound 18d, 44 g, 15.77 mmol) in DMA (156 mL). The reaction mixture was heated at 100° C. for 18 hours. The reaction was diluted with EA (200 mL), washed with water (100 mL) and brine (50 mL). The organic layer was concentrated, and the residue was purified by silica gel column to give compound 18e (23 g) as a white solid. LCMS(M+H)⁺: 179.

### Step 5: Preparation of tert-butyl-N-[(3R,5S)-1-(4-cyano-1,3-benzothiazol-7-yl)-5-methyl-3-piperidyl]carbamate (Compound 18f)

A mixture of 7-fluoro-1,3-benzothiazole-4-carbonitrile (compound 18e, 70.0 mg, 0.390 mmol), tert-butyl N-[(3R,5S)-5-methyl-3-piperidyl]carbamate (101.0 mg, 0.470 mmol) and DIPEA (0.21 mL, 1.18 mmol) in DMA (5 mL) was stirred at 130° C. for 16 hours. Water (50 ml) was added and the mixture was extracted with EA (50 mL), the organic layer was concentrated and the residue was purified by preparative TLC (EA:PE=1:2, Rf: 0.3) to afford compound 18f (120 mg) as a yellow gum. LCMS(M+H)⁺: 373.

### Step 6: Preparation of 7-[(3R,5S)-3-amino-5-methyl-1-piperidyl]-1,3-benzothiazole-4-carbonitrile (Compound 18h)

To a solution of tert-butyl N-[(3R,5S)-1-(4-cyano-1,3-benzothiazol-7-yl)-5-methyl-3-piperidyl]carbamate (compound 18f, 120.0 mg, 0.320 mmol) in DCM (5 mL) was added trifluoroacetic acid (1.85 mL, 24 mmol) under ice-bath. After the mixture was stirred at 25° C. for 1 hour, it was concentrated in vacuum. Ice-water (20 mL) was added to the residue, and the mixture was neutralized with sat. Na₂CO₃ (20 mL), extracted with DCM (50 mL). The organic layer was dried and concentrated to afford compound 18h (80 mg) as a yellow gum. LCMS(M+H)⁺: 273.

### Step 7: Preparation of 5-(4-methylpiperazin-1-yl)pyridine-2-carbaldehyde (Compound 18k)

To a solution of 5-fluoro-2-formylpyridine (compound 18j, 160.0 mg, 1.28 mmol) in DMA (5 mL) was added 1-methylpiperazine (192.15 mg, 1.92 mmol) and K₂CO₃ (530.3 mg, 3.84 mmol). The mixture was stirred at 120° C. for 12 hours. After added water (50 mL), the mixture was then extracted with EtOAc (50 mL). The organic layer was concentrated in vacuum, and the residue was purified by flash column chromatography to afford compound 18k (200 mg) as a white solid. LCMS(M+H)⁺: 206.

### Step 8: Preparation of 7-[(3S,5R)-3-methyl-5-[[5-(4-methylpiperazin-1-yl)-2-pyridyl]methylamino]-1-piperidyl]-1,3-benzothiazole-4-carbonitrile (Example 18)

To a solution of 7-[(3R,5S)-3-amino-5-methyl-1-piperidyl]-1,3-benzothiazole-4-carbonitrile (compound 18h, 30.0 mg, 0.110 mmol) in methanol (2 mL) was added 5-(4-methylpiperazin-1-yl)pyridine-2-carbaldehyde (compound 18k, 45.22 mg, 0.220 mmol) and acetic acid (0.66 mg, 0.010 mmol). After the mixture was stirred at 25° C. for 15 hours, sodium borohydride (20.84 mg, 0.550 mmol) was added. After stirred at 25° C. for 0.5 hours, the reaction mixture was added with water (50 mL), then extracted with DCM (50 mL). The organic layer was concentrated and the residue was purified by preparative HPLC to afford Example 18 (18 mg) as a colorless gum. LCMS(M+H)⁺: 462, ¹H NMR (400 MHz, METHANOL-d4) δ ppm: 9.40 (s, 1H), 8.36 (d, J=2.8 Hz, 1H), 7.88 (d, J=8.0 Hz, 1H), 7.48-7.43 (m, 1H), 7.43-7.37 (m, 1H), 7.15 (d, J=8.3 Hz, 1H), 4.31 (d, J=2.0 Hz, 2H), 4.18-4.08 (m, 1H), 3.79 (br d, J=9.0 Hz, 1H), 3.47-3.37 (m, 5H), 3.09-3.00 (m, 4H), 2.93 (t, J=11.4 Hz, 1H), 2.66 (s, 3H), 2.62-2.53 (m, 1H), 2.39 (br d, J=12.5 Hz, 1H), 2.07-1.92 (m, 1H), 1.31 (q, J=12.0 Hz, 1H), 1.08 (d, J=6.5 Hz, 3H).

### Example 21

### 4-[(3S,5R)-3-methyl-5-[(4-piperazin-1-ylphenyl)methylamino]-1-piperidyl]pyrazolo[1,5-a]pyridine-7-carbonitrile

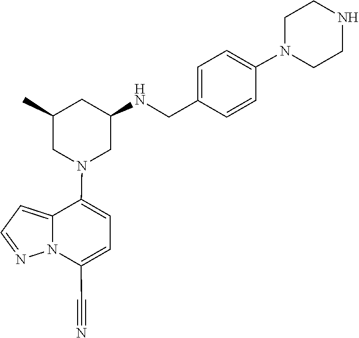 [SMILES: C[C@H]1C[C@@H](NCc2ccc(N3CCNCC3)cc2)CN(c2ccc(C#N)n3nccc23)C1]

The title compound was prepared in analogy to the preparation of Example 1 by using 4-chloropyrazolo[1,5-a]pyridine-7-carbonitrile instead of compound 1a. Example 21 was obtained as a light brown solid (150 mg). LCMS(M+H)⁺: 430, ¹H NMR (400 MHz, METHANOL-d₄) δ ppm: 8.07 (d, J=2.3 Hz, 1H), 7.48 (dd, J=8.3, 16.8 Hz, 3H), 7.12 (d, J=8.8 Hz, 2H), 6.83 (d, J=2.4 Hz, 1H), 6.68 (d, J=7.9 Hz, 1H), 4.40-4.19 (m, 2H), 3.86 (br d, J=8.8 Hz, 1H), 3.66-3.53 (m, 1H), 3.50-3.45 (m, 4H), 3.43-3.37 (m, 5H), 2.89 (t, J=11.5 Hz, 1H), 2.59 (t, J=12.0 Hz, 1H), 2.48 (br d, J=12.1 Hz, 1H), 2.09 (br d, J=5.0 Hz, 1H), 1.43-1.29 (m, 1H), 1.12 (d, J=6.6 Hz, 3H).

### Example 22

### (3R,5S)-1-(8-chloro-5-quinolyl)-5-methyl-N-[(4-piperazin-1-ylphenyl)methyl]piperidin-3-amine

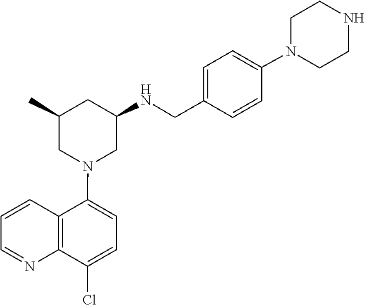 [SMILES: C[C@H]1C[C@@H](NCc2ccc(N3CCNCC3)cc2)CN(c2ccc(Cl)c3ncccc23)C1]

The title compound was prepared in analogy to the preparation of Example 1 by using 5-bromo-8-chloro-quinoline instead of compound 1a. Example 22 was obtained as a light brown solid (55 mg). LCMS(M+H)⁺: 450, ¹H NMR (400 MHz, METHANOL-d₄) δ ppm: 9.00 (dd, J=1.7, 4.3 Hz, 1H), 8.70 (dd, J=1.7, 8.5 Hz, 1H), 7.90 (d, J=8.2 Hz, 1H), 7.72 (dd, J=4.3, 8.5 Hz, 1H), 7.46 (d, J=8.8 Hz, 2H), 7.32 (d, J 8.2 Hz, 1H), 7.12 (d, J 8.8 Hz, 2H), 4.28 (q, J=13.0 Hz, 2H), 3.79-3.66 (m, 2H), 3.55-3.45 (m, 4H), 3.43-3.37 (m, 5H), 2.91-2.77 (m, 1H), 2.58-2.43 (m, 2H), 2.25-2.17 (m, 1H), 1.43-1.29 (m, 1H), 1.10 (d, J=6.6 Hz, 3H).

### Example 23

### 5-[(3S,5R)-3-methyl-5-(3-piperazin-1-yl-5,7-dihydropyrrolo[3,4-b]pyridin-6-yl)-1-piperidyl]quinoline-8-carbonitrile

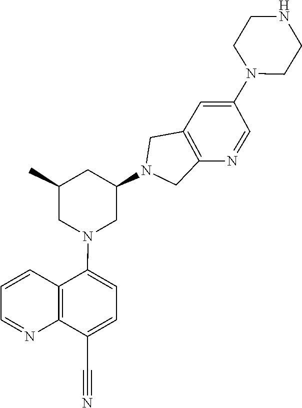 [SMILES: C[C@H]1C[C@@H](N2Cc3cc(N4CCNCC4)cnc3C2)CN(c2ccc(C#N)c3ncccc23)C1]

The title compound was prepared according to the following scheme:

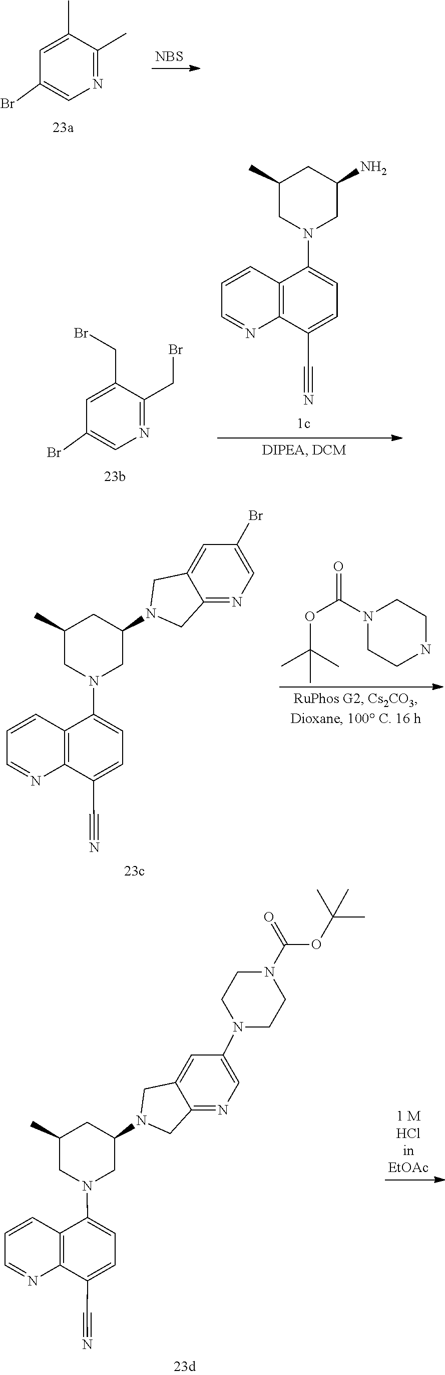

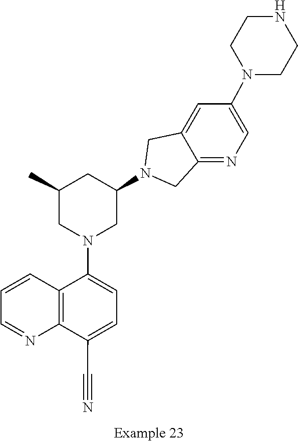 [SMILES: C[C@H]1C[C@@H](N2Cc3cc(N4CCNCC4)cnc3C2)CN(c2ccc(C#N)c3ncccc23)C1]

### Step 1: Preparation of 5-bromo-2,3-bis(bromomethyl)pyridine (Compound 23b)

A mixture of 5-bromo-2,3-dimethylpyridine (compound 23a, 1 g, 5.37 mmol), NBS (2.1 g, 11.8 mmol) and benzoyl peroxide (130 mg, 537 μmol) in CCl₄ (50 mL) was stirred at 80° C. for 6 hours, then the reaction mixture was concentrated and the crude residue was purified by flash chromatography (silica gel, 40 g, 0% to 10% EtOAc in hexanes) to give compound 23b as light orange oil (1 g). LCMS(M+H)⁺: 342.

### Step 2: Preparation of 5-[(3S,5R)-3-methyl-5-(3-piperazin-1-yl-5,7-dihydropyrrolo[3,4-b]pyridin-6-yl)-1-piperidyl]quinoline-8-carbonitrile (Example 23)

The title compound was prepared in analogy to the preparation of Example 12 by using compound 23b instead of 4-bromo-1,2-bis(bromomethyl)benzene. Example 23 was obtained as an orange solid (22 mg). LCMS (M+H)⁺: 454, ¹H NMR (400 MHz, METHANOL-d₄) δ ppm: 9.09 (dd, J=1.5, 4.5 Hz, 1H), 8.81 (dd, J=1.7, 8.6 Hz, 1H), 8.41 (d, J=2.6 Hz, 1H), 8.30 (d, J=8.1 Hz, 1H), 7.83 (dd, J=4.6, 8.6 Hz, 1H), 7.69 (s, 1H), 7.49 (d, J=8.1 Hz, 1H), 4.97 (br s 2H), 4.87 (br s, 2H), 4.17 (br t, J=11.2 Hz, 1H), 4.03 (br d, J=12.1 Hz, 1H), 3.67-3.50 (m, 5H), 3.47-3.39 (m, 4H), 3.25-3.11 (m, 1H), 2.74-2.54 (m, 2H), 2.33 (br s, 1H), 1.53 (q, J=12.1 Hz, 1H), 1.15 (d, J=6.6 Hz, 3H).

### Example 24

### 5-[cis-3-methyl-5-(6-piperazin-1-yl-3,4-dihydro-1H-isoquinolin-2-yl)-1-piperidyl]quinoline-8-carbonitrile

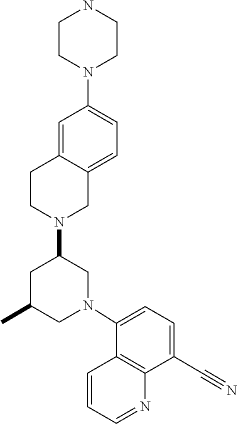

The title compound was prepared according to the following scheme:

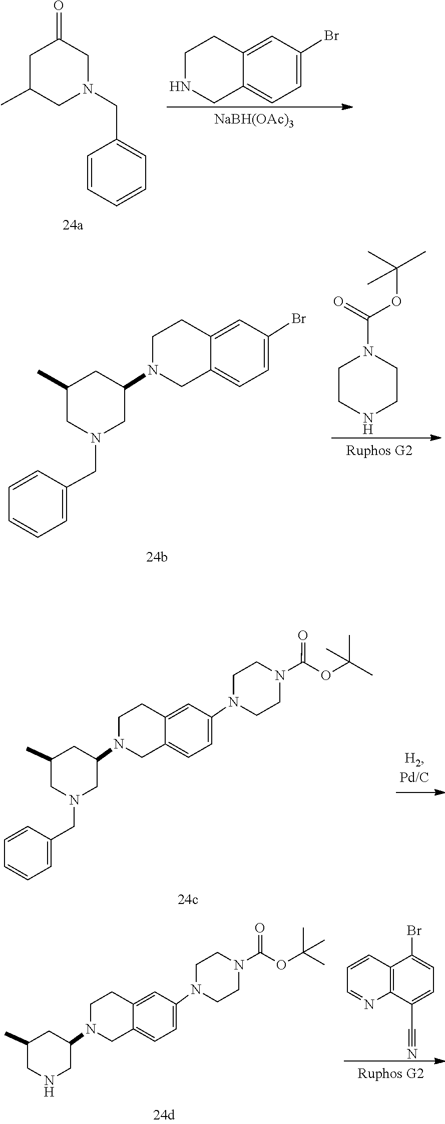

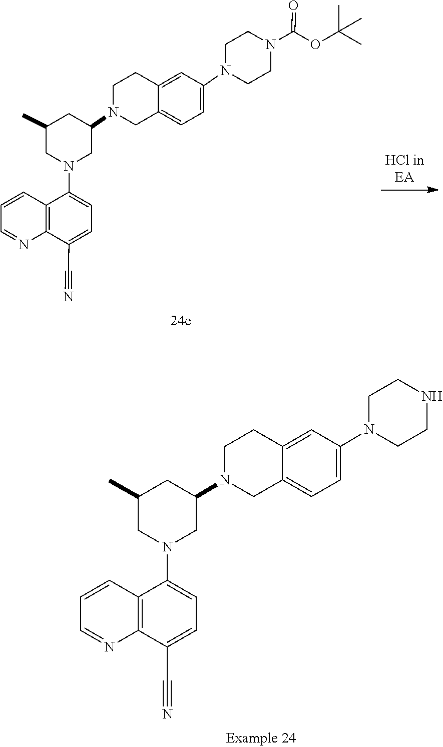 [SMILES: C[C@H]1C[C@@H](N2CCc3cc(N4CCN(C(=O)OC(C)(C)C)CC4)ccc3C2)CN(c2ccc(C#N)c3ncccc23)C1 ; C[C@H]1C[C@@H](N2CCc3cc(N4CCNCC4)ccc3C2)CN(c2ccc(C#N)c3ncccc23)C1]

### Step 1: Preparation of 2-(cis-1-benzyl-5-methylpiperidin-3-yl)-6-bromo-1,2,3,4-tetrahydroisoquinoline (Compound 24b)

A mixture of 1-benzyl-5-methylpiperidin-3-one (compound 24a, 1 g, 4.92 mmol), 6-bromo-1,2,3,4-tetrahydroisoquinoline (1.04 g, 4.92 mmol) and sodium triacetoxyborohydride (3.13 g, 14.8 mmol) in DCM (15 mL) was stirred at rt for 2 hours. The reaction mixture was diluted with EA, washed with water and brine. The organic layer was concentrated and the residue was purified by preparative HPLC to give cis-isomer compound 24b (150 mg) and its trans-isomer 24b′ (500 mg) as a colorless oil. Cis-isomer: LCMS(M+H)⁺: 399, ¹H NMR (400 MHz, METHANOL-d₄) δ ppm: 7.39-7.23 (m, 7H), 6.99 (d, J=8.2 Hz, 1H), 3.84-3.70 (m, 2H), 3.61 (s, 2H), 3.19 (br d, J=10.4 Hz, 1H), 2.84-2.74 (m, 3H), 2.21 (t, J=7.6 Hz, 1H), 2.10-2.02 (m, 2H), 1.96 (t, J=10.8 Hz, 1H), 1.76 (br dd, J=4.5, 11.2 Hz, 1H), 1.66-1.56 (m, 1H), 1.26 (t, J=7.1 Hz, 1H), 1.11-0.99 (m, 1H), 0.98-0.88 (m, 3H).

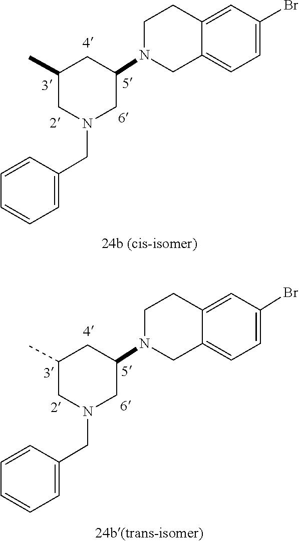 [SMILES: C[C@H]1C[C@@H](N2CCc3cc(Br)ccc3C2)CN(Cc2ccccc2)C1 ; C[C@@H]1C[C@@H](N2CCc3cc(Br)ccc3C2)CN(Cc2ccccc2)C1]

For compound 24b, the NOESY correlation of C3′-H and C5′-H was observed. For compound 24b′, the NOESY correlation of C3′-H and C5′-H was not observed.

### Step 2: Preparation of tert-butyl 4-[2-(cis-1-benzyl-5-methyl-3-piperidyl)-3,4-dihydro-1H-isoquinolin-6-yl]piperazine-1-carboxylate (Compound 24c)

A mixture of 2-(cis-1-benzyl-5-methylpiperidin-3-yl)-6-bromo-1,2,3,4-tetrahydroisoquinoline (compound 24b, 150 mg, 376 μmol, tert-butyl piperazine-1-carboxylate (140 mg, 751 μmol, Cs₂CO₃ (367 mg, 1.13 mmol) and RuPhos G2 (29.2 mg, 37.6 μmol in dioxane (5 mL) was charged with N₂. The reaction mixture was heated at 100° C. for 16 hours. After cooling, the undissolved material was filtered off and washed with EA (10 mL). The combined filtrates were concentrated and purified by flash chromatography (eluting with 10% to 80% EtOAc in hexanes) to give compound 24c as a colorless oil (100 mg). LCMS(M+H)⁺: 505.

### Step 3: Preparation of tert-butyl 4-[2-(cis-5-methylpiperidin-3-yl)-1,2,3,4-tetrahydroisoquinolin-6-yl]piperazine-1-carboxylate (Compound 24d)

A mixture of 4-[2-(cis-1-benzyl-5-methyl-3-piperidyl)-3,4-dihydro-1H-isoquinolin-6-yl]piperazine-1-carboxylate (compound 24c, 100 mg, 198 μmol and Pd—C (50 mg) in MeOH/EtOAc (1:2) (15 mL) under hydrogen balloon was stirred at rt for 24 hours. The undissolved material was filtered off, and the filtrate was concentrated to give compound 24d as a colorless oil (80 mg). LCMS(M+H)⁺: 415.

### Step 4: Preparation of tert-butyl 4-[2[-cis-1-(8-cyano-5-quinolyl)-5-methyl-3-piperidyl]-3,4-dihydro-1H-isoquinolin-6-yl]piperazine-1-carboxylate (Compound 24e)

A mixture of 5-bromoquinoline-8-carbonitrile (101 mg, 434 μmol, tert-butyl 4-[2-(cis-5-methylpiperidin-3-yl)-1,2,3,4-tetrahydroisoquinolin-6-yl]piperazine-1-carboxylate (compound 24d, 90 mg, 217 μmol, RuPhos G2 (16.9 mg, 21.7 μmol) and Cs₂CO₃ (106 mg, 326 μmol in dioxane (5 mL) was charged with N₂. The mixture was heated at 80° C. overnight. After cooling, the solid was filtered off and washed with EA (10 mL). The combined filtrates were concentrated and the residue was purified by preparative HPLC to give compound 24e as a light yellow solid (22 mg). LCMS(M+H)⁺: 567.

### Step 5: Preparation of 5-[cis-3-methyl-5-(6-piperazin-1-yl-3,4-dihydro-1H-isoquinolin-2-yl)-1-piperidyl]quinoline-8-carbonitrile (Example 24)

A mixture of tert-butyl 4-[2-[cis-1-(8-cyano-5-quinolyl)-5-methyl-3-piperidyl]-3,4-dihydro-1H-isoquinolin-6-yl]piperazine-1-carboxylate (compound 24e, 20 mg, 35.3 μmol) in 1 M HCl in EA (5 mL) was stirred at rt for 16 hours. The solution was then concentrated to give Example 24 as an orange solid (18 mg). LCMS(M+H)⁺: 467. ¹H NMR (400 MHz, METHANOL-d₄) δ ppm: 9.10 (d, J=4.6 Hz, 1H), 8.92-8.76 (m, 1H), 8.30 (dd, J=2.1, 7.9 Hz, 1H), 7.85 (ddd, J=4.7, 8.8, 13.8 Hz, 1H), 7.49 (dd, J=1.7, 8.2 Hz, 1H), 7.19 (t, J=6.9 Hz, 1H), 7.02 (dd, J=2.5, 8.5 Hz, 1H), 6.95 (br s, 1H), 4.56 (br s, 2H), 4.12-3.87 (m, 3H), 3.64-3.51 (m, 2H), 3.51-3.35 (m, 9H), 3.29-3.10 (m, 2H), 2.71-2.60 (m, 1H), 2.60-2.45 (m, 1H), 2.31 (br d, J=6.8 Hz, 1H), 1.69-1.48 (m, 1H), 1.15 (d, J=6.6 Hz, 3H).

### Example 25

### 5-[(3S,5R)-3-methyl-5-[(5-piperazin-1-ylpyrimidin-2-yl)methylamino]-1-piperidyl]quinoline-8-carbonitrile

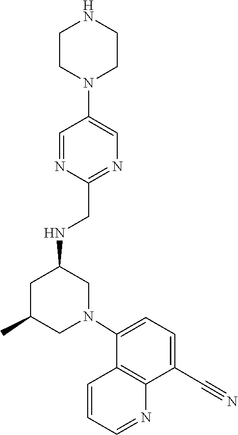 [SMILES: C[C@H]1C[C@@H](NCc2ncc(N3CCNCC3)cn2)CN(c2ccc(C#N)c3ncccc23)C1]

The title compound was prepared in analogy to the preparation of Example 4 by using 5-bromopyrimidine-2-carbaldehyde instead of 4-bromo-2-fluorobenzaldehyde. Example 25 was obtained as an orange solid (14 mg). LCMS(M+H)⁺: 443, ¹H NMR (400 MHz, METHANOL-d₄) δ ppm: 9.08 (dd, J=1.5, 4.4 Hz, 1H), 8.76 (dd, J=1.5, 8.6 Hz, 1H), 8.64 (s, 2H), 8.27 (d, J 7.9 Hz, 1H), 7.81 (dd, J=4.5, 8.6 Hz, 1H), 7.42 (d, J=8.2 Hz, 1H), 4.65-4.47 (m, 2H), 3.98 (br d, J=10.9 Hz, 1H), 3.92-3.82 (m, 1H), 3.67-3.59 (m, 4H), 3.55-3.35 (m, 5H), 3.05 (t, J=11.1 Hz, 1H), 2.63 (t, J=11.7 Hz, 1H), 2.52 (br d, J=12.3 Hz, 1H), 2.27 (br d, J=3.9 Hz, 1H), 1.42 (q, J=12.0 Hz, 1H), 1.12 (d, J=6.6 Hz, 3H).

### Example 26

### 5-[(3S,5R)-3-methyl-5-[1-(4-piperazin-1-ylphenyl)ethylamino]-1-piperidyl]quinoline-8-carbonitrile

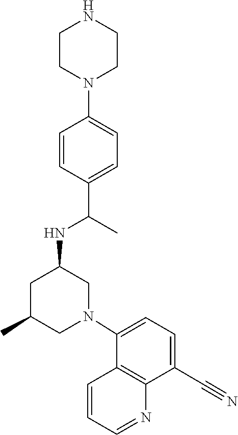 [SMILES: CC(N[C@@H]1C[C@H](C)CN(c2ccc(C#N)c3ncccc23)C1)c1ccc(N2CCNCC2)cc1]

The title compound was prepared according to the following scheme:

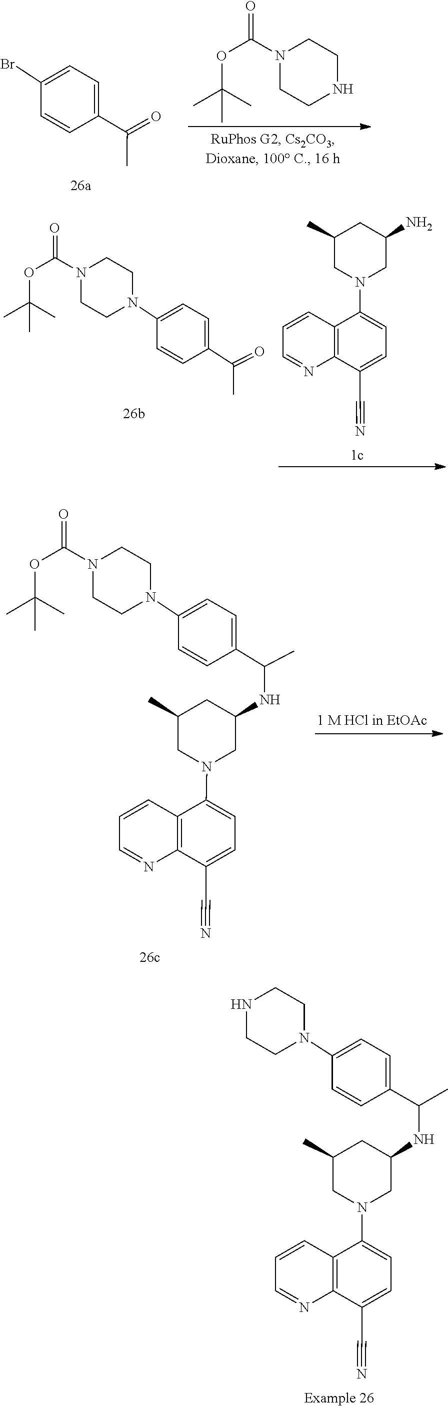

### Step 1: Preparation of tert-butyl 4-(4-acetylphenyl)piperazine-1-carboxylate (Compound 26b)

A mixture of 1-(4-bromophenyl)ethan-1-one (compound 26a, 400 mg, 2.01 mmol), tert-butyl piperazine-1-carboxylate (749 mg, 4.02 mmol), RuPhos G2 (156 mg, 201 μmol and Cs₂CO₃ (1.31 g, 4.02 mmol) in dioxane (15 mL) was charged with N₂. The mixture was heated at 100° C. for 16 hours. After cooling, the solid was filtered off and washed with EA (10 mL). The combined filtrate was concentrated and the residue was purified by silica gel to give compound 26b as a white solid (400 mg). LCMS(M+H)⁺: 305.

### Step 2: Preparation of tert-butyl 4-(4-(1-(((3R,5S)-1-(8-cyanoquinolin-5-yl)-5-methylpiperidin-3-yl)amino)ethyl)phenyl)piperazine-1-carboxylate (Compound 26b)

A mixture of 5-(3R,5S)-3-amino-5-methylpiperidin-1-yl)quinoline-8-carbonitrile (40 mg, 150 μmol), tert-butyl 4-(4-acetylphenyl)piperazine-1-carboxylate (compound 26b, 68.6 mg, 225 μmol and titanium (IV) isopropoxide (220 μL, 751 μmol), 4 Å molecular sieve (500 mg) in toluene (10 mL) was stirred at 100° C. for 16 hours. After the reaction mixture was concentrated, the residue was dissolved in DCM (10 mL) to form a solution, which was added sodium triacetoxyborohydride (159 mg, 751 μmol) and stirred at rt for 30 mins. The solid was filtered off and the filtrate was concentrated. The residue was purified by HPLC to give compound 26c as a light yellow solid (16 mg). LCMS(M+H)⁺: 555.

### Step 3: Preparation of 5-[(3S,5R)-3-methyl-5-[1-(4-piperazin-1-ylphenyl)ethylamino]-1-piperidyl]quinoline-8-carbonitrile (Example 26)

A mixture of tert-butyl 4-(4-(1-(((3R,5S)-1-(8-cyanoquinolin-5-yl)-5-methylpiperidin-3-yl)amino)ethyl)phenyl)piperazine-1-carboxylate (compound 26c, 16 mg, 28.8 μmol) in HCl in EA (1M, 5 mL) was stirred at rt for 16 hours. The mixture was concentrated to give Example 26 as an orange solid (15 mg). LCMS(M+H)⁺: 455, ¹H NMR (400 MHz, METHANOL-d₄) δ ppm: 9.06 (ddd, J=1.6, 4.4, 13.3 Hz, 1H), 8.48 (dd, J=1.5, 8.6 Hz, 1H), 8.21 (dd, J=2.0, 8.0 Hz, 1H), 7.73 (ddd, J=4.5, 8.7, 13.5 Hz, 1H), 7.50 (d, J=8.8 Hz, 1H), 7.42 (d, J=8.8 Hz, 1H), 7.36-7.26 (m, 1H), 7.11 (t, J=8.6 Hz, 2H), 4.64-4.54 (m, 1H), 3.92 (br d, J=8.9 Hz, 0.5H), 3.57-3.38 (m, 10.5H), 3.00-2.81 (m, 1H), 2.69-2.44 (m, 1.5H), 2.32 (br d, J=13.0 Hz, 0.5H), 2.12 (br s, 1H), 1.70 (dd, J=6.8, 13.1 Hz, 3H), 1.41-1.28 (m, 1H), 1.07 (t, J=6.6 Hz, 3H).

### Example 28

### 5-[(3S,5R)-3-methyl-5-[[2-[(3R)-3-methylpiperazin-1-yl]pyrimidin-5-yl]methylamino]-1-piperidyl]quinoline-8-carbonitrile

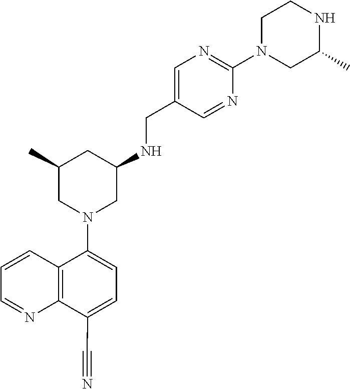 [SMILES: C[C@H]1C[C@@H](NCc2cnc(N3CCN[C@H](C)C3)nc2)CN(c2ccc(C#N)c3ncccc23)C1]

The title compound was prepared in analogy to the preparation of Example 17 by using tert-butyl (3R)-3-methylpiperazine-1-carboxylate instead of tert-butyl piperazine-1-carboxylate. Example 28 was obtained as an orange solid (83 mg). LCMS(M+H)⁺: 457, ¹H NMR (400 MHz, METHANOL-d₄) δ ppm: 9.13 (dd, J=1.5, 4.7 Hz, 1H), 8.93 (dd, J=1.6, 8.6 Hz, 1H), 8.65 (s, 2H), 8.34 (d, J=8.1 Hz, 1H), 7.92 (dd, J=4.8, 8.6 Hz, 1H), 7.49 (d, J=8.2 Hz, 1H), 4.87 (br s, 1H), 4.42-4.24 (m, 2H), 4.03 (br d, J=14.1 Hz, 1H), 3.91-3.82 (m, 1H), 3.58-3.35 (m, 5H), 3.25-3.12 (m, 2H), 3.04 (t, J=11.1 Hz, 1H), 2.67 (t, J=11.7 Hz, 1H), 2.55 (br d, J=12.3 Hz, 1H), 2.35-2.21 (m, 1H), 1.49-1.27 (m, 4H), 1.13 (d, J=6.6 Hz, 3H).

### Example 29

### 5-[(3S,5R)-3-methyl-5-[[2-[(3S)-3-methylpiperazin-1-yl]pyrimidin-5-yl]methylamino]-1-piperidyl]quinoline-8-carbonitrile

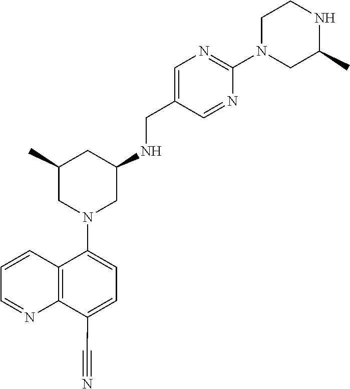 [SMILES: C[C@H]1C[C@@H](NCc2cnc(N3CCN[C@@H](C)C3)nc2)CN(c2ccc(C#N)c3ncccc23)C1]

The title compound was prepared in analogy to the preparation of Example 17 by using tert-butyl (3S)-3-methylpiperazine-1-carboxylate instead of tert-butyl piperazine-1-carboxylate. Example 29 was obtained as an orange solid (63 mg). LCMS(M+H)⁺: 457, ¹H NMR (400 MHz, METHANOL-d₄) δ ppm: 9.14 (dd, J=1.5, 4.8 Hz, 1H), 8.97 (dd, J=1.5, 8.6 Hz, 1H), 8.65 (s, 2H), 8.35 (d, J=8.2 Hz, 1H), 7.94 (dd, J=4.8, 8.6 Hz, 1H), 7.50 (d, J=8.2 Hz, 1H), 4.87 (br s, 1H), 4.43-4.25 (m, 2H), 4.03 (br d, J=9.9 Hz, 1H), 3.92-3.83 (m, 1H), 3.58-3.35 (m, 4H), 3.25-3.13 (m, 2H), 3.05 (t, J=11.1 Hz, 1H), 2.68 (t, J=11.6 Hz, 1H), 2.56 (br d, J=12.1 Hz, 1H), 2.28 (br d, J=6.4 Hz, 1H), 1.45-1.35 (m, 4H), 1.13 (d, J=6.7 Hz, 3H).

### Example 30

### 5-[(3S,5R)-3-methyl-5-(5,6,7,8-tetrahydro-1,6-naphthyridin-2-ylmethylamino)-1-piperidyl]quinoline-8-carbonitrile

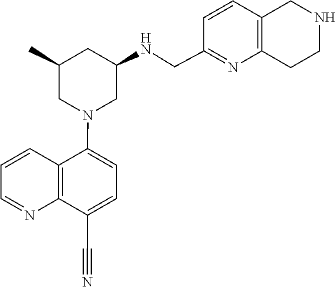 [SMILES: C[C@H]1C[C@@H](NCc2ccc3c(n2)CCNC3)CN(c2ccc(C#N)c3ncccc23)C1]

The title compound was prepared according to the following scheme:

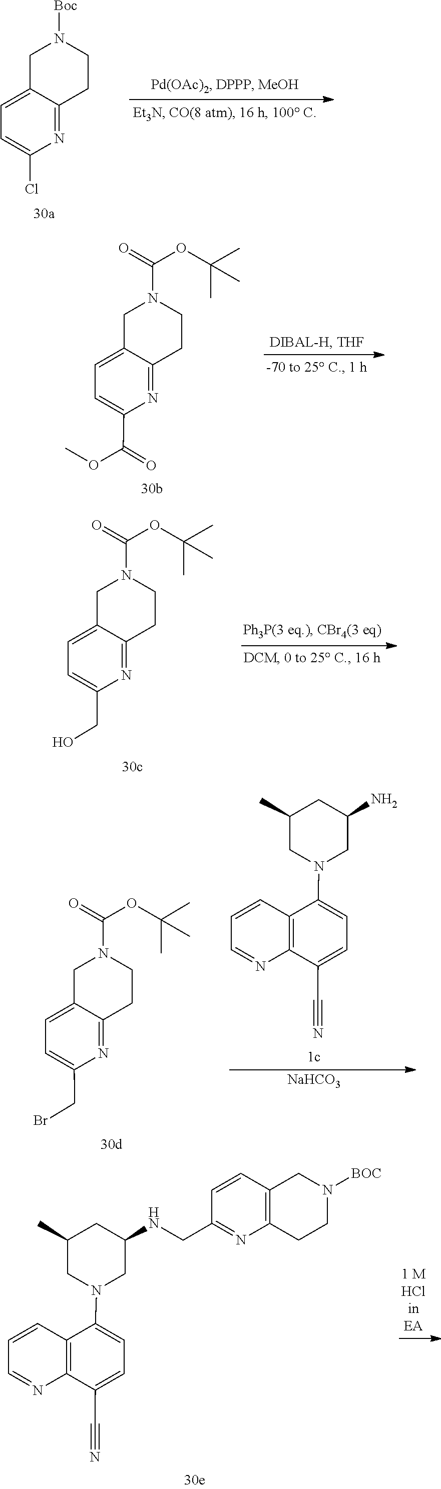

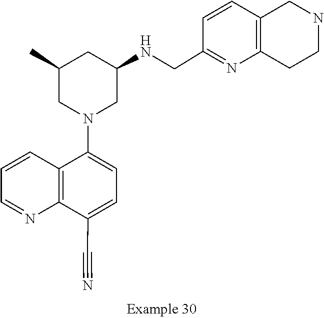

### Step 1: Preparation of O6-tert-butyl O2-methyl 7,8-dihydro-5H-1,6-naphthyridine-2,6-dicarboxylate (Compound 30b)

To a round bottom flask were added tert-butyl 2-chloro-7,8-dihydro-5H-1,6-naphthyridine-6-carboxylate (compound 30a, 950.0 mg, 3.54 mmol), Pd(AcO)₂ (158.73 mg, 0.710 mmol), DPPP (291.6 mg, 0.710 mmol), Et₃N (1788.54 mg, 17.68 mmol) and methanol (50 mL). The mixture was stirred at 100° C. for 16 hours under CO (6080 mmHg). Then the mixture was concentrated, and the residue was purified by silica gel column chromatography (EA/PE=1/3 to 1/1) to afford compound 30b as a light yellow solid (930 mg). LCMS(M+H)⁺: 293.

### Step 2: Preparation of tert-butyl 2-(hydroxymethyl)-7,8-dihydro-5H-1,6-naphthyridine-6-carboxylate (Compound 30c)

To a solution of O6-tert-butyl O2-methyl 7,8-dihydro-5H-1,6-naphthyridine-2,6-dicarboxylate (compound 30b, 930.0 mg, 3.18 mmol) in THF (50 mL) at −70° C. under N₂ was added DIBAL-H (15.91 mL, 15.91 mmol). The mixture was stirred at −70° C. for 0.5 h and then warmed slowly to 25° C. The mixture was stirred at 25° C. for 1 h, and then quenched with saturated aqueous sodium potassium tartrate (50 mL). The resulting mixture was stirred at 25° C. for 1 h and separated. The aqueous phase was extracted with DCM (30 mL) twice. The organic phase was combined, washed with brine (50 mL), dried over Na₂SO₄, and concentrated. The residue was purified by silica gel chromatography (DCM/MeOH=20/1) to afford compound 30c as colorless oil (320 mg). LC/MS(M+H)⁺: 265.

### Step 3: Preparation of tert-butyl 2-(bromomethyl)-7,8-dihydro-5H-1,6-naphthyridine-6-carboxylate (Compound 30d)

A round bottom flask was charged with tert-butyl 2-(hydroxymethyl)-7,8-dihydro-5H-1,6-naphthyridine-6-carboxylate (320.0 mg, 1.21 mmol), triphenylphosphine (960.0 mg, 3.66 mmol) and DCM (40 mL). Carbon tetrabromide (1208 mg, 3.65 mmol) was added at 0° C. under N₂. After warmed to 25° C. and stirred at 25° C. for 16 hours, the mixture was concentrated, and the residue was purified by silica gel column chromatography (PE/EA=2/1) to afford compound 30d (260 mg). LC/MS(M+H)⁺: 327.

### Step 4: Preparation of tert-butyl 2-((((3R,5S)-1-(8-cyanoquinolin-5-yl)-5-methylpiperidin-3-yl)amino)methyl)-7,8-dihydro-5H-1,6-naphthyridine-6-carboxylate (Compound 30e)

A mixture of 5-((3R,5S)-3-amino-5-methyl piperidin-1-yl)quinoline-8-carbonitrile hydrochloride (compound 1c, 50 mg, 165 μmol), tert-butyl 2-(bromomethyl)-7,8-dihydro-5H-1,6-naphthyridine-6-carboxylate (compound 30d, 54 mg, 165 μmol and sodium bicarbonate (41.6 mg, 495 μmol in DMF (5 mL) was stirred at 60° C. for 16 hours. The solid was filtered off and the filtrate was purified by preparative HPLC to give compound 30e as a light yellow solid (30 mg). LC/MS(M+H)⁺: 513.

### Step 5: Preparation of 5-[(3S,5R)-3-methyl-5-(5,6,7,8-tetrahydro-1,6-naphthyridin-2-ylmethylamino)-1-piperidyl]quinoline-8-carbonitrile (Example 30)

A mixture of tert-butyl 24(43R,5S)-1-(8-cyanoquinolin-5-yl)-5-methylpiperidin-3-yl)amino)methyl)-7,8-dihydro-5H-1,6-naphthyridine-6-carboxylate (compound 30e, 20 mg, 39 μmol in 1 M HCl in EA (10 mL) was stirred at rt for 16 hours, then the reaction was concentrated to give Example 30 as an orange solid (20 mg). LCMS(M+H)⁺: 413, ¹H NMR (400 MHz, DMSO-d₆) δ ppm 9.80 (br s, 2H), 9.64 (br s, 2H), 9.07 (dd, J=1.5, 4.2 Hz, 1H), 8.50 (d, J=7.1 Hz, 1H), 8.29 (d, J=7.9 Hz, 1H), 7.85-7.66 (m, 2H), 7.49 (d, J=7.9 Hz, 1H), 7.30 (d, J=8.1 Hz, 1H), 4.50-4.28 (m, 4H), 3.96 (br d, J=12.5 Hz, 1H), 3.70 (br s, 1H), 3.54-3.38 (m, 3H), 3.22-3.10 (m, 2H), 2.95 (t, J=11.3 Hz, 1H), 2.45-2.32 (m, 1H), 2.06 (br s, 1H), 1.39 (q, J=12.1 Hz, 1H), 1.00 (d, J=6.6 Hz, 3H).

### Example 31

### 5-[(3S,5R)-3-methyl-5-[(5-piperazin-1-ylpyrazin-2-yl)methylamino]-1-piperidyl]quinoline-8-carbonitrile

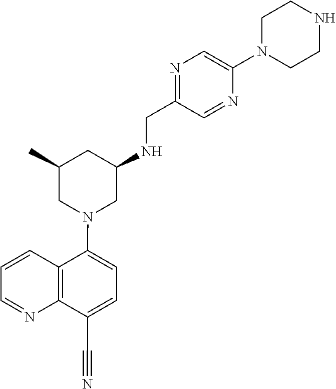 [SMILES: C[C@H]1C[C@@H](NCc2cnc(N3CCNCC3)cn2)CN(c2ccc(C#N)c3ncccc23)C1]

The title compound was prepared according to the following scheme:

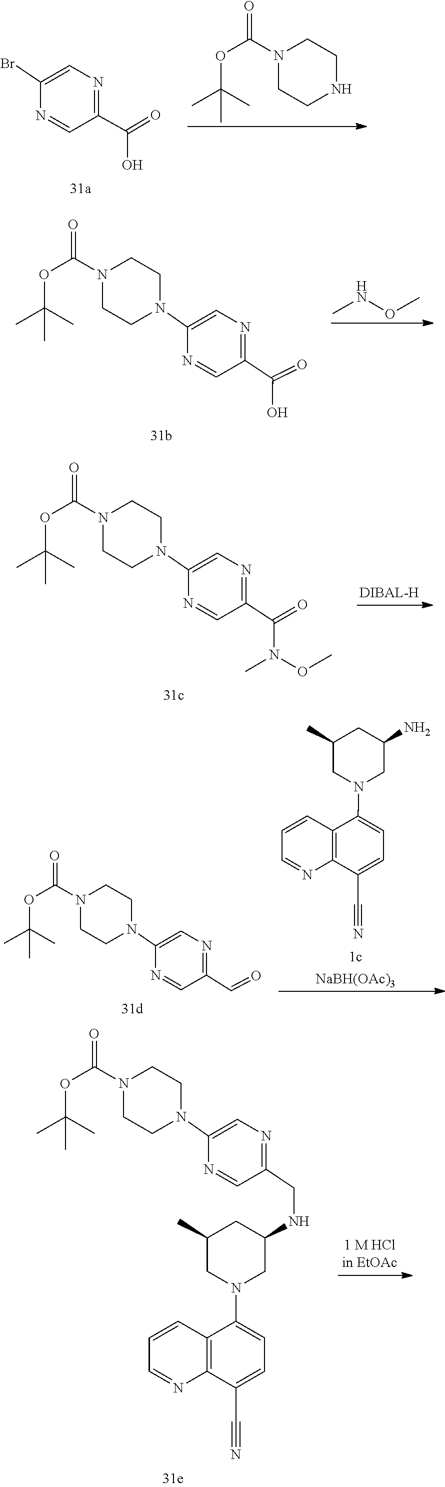

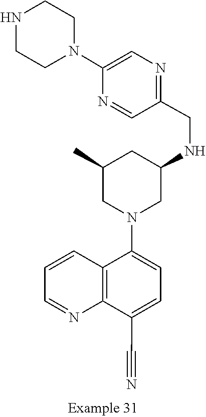 [SMILES: C[C@H]1C[C@@H](NCc2cnc(N3CCNCC3)cn2)CN(c2ccc(C#N)c3ncccc23)C1]

### Step 1: Preparation of 5-(4-(tert-butoxycarbonyl)piperazin-1-yl)pyrazine-2-carboxylic acid (Compound 31b)

A mixture of 5-bromopyrazine-2-carboxylic acid (compound 31a, 1 g, 4.93 mmol), tert-butyl piperazine-1-carboxylate (1.84 g, 9.85 mmol) and DIPEA (4.3 ml, 24.6 mmol) in DMSO (20 mL) was stirred at 110° C. for 2 hours. After cooling to rt, white solid was precipitated and collected to afford compound 31b (1.5 g). LCMS(M+H)⁺: 309.

### Step 2: Preparation of tert-butyl 4-(5-(methoxy(methyl)carbamoyl)pyrazin-2-yl)piperazine-1-carboxylate (Compound 31c)

A mixture of 5-(4-(tert-butoxycarbonyl)piperazin-1-yl)pyrazine-2-carboxylic acid (compound 31b, 1 g, 3.24 mmol), N,O-dimethylhydroxylamine hydrochloride (949 mg, 9.73 mmol), HATU (1.85 g, 4.86 mmol) and DIPEA (2.83 ml, 16.2 mmol) in DMF (20 mL) was stirred at rt for 2 hours. The mixture was diluted with EA, washed with water and brine. The organic layer was dried and concentrated, and the crude residue was purified by flash chromatography (silica gel, 0% to 40% EtOAc in hexanes) to give compound 31c as a light yellow solid (0.9 g). LCMS(M+H)⁺: 352.

### Step 3: Preparation of tert-butyl 4-(5-formylpyrazin-2-yl)piperazine-1-carboxylate (Compound 31d)

DIBAL-H (1.71 ml, 1.71 mmol) was added dropwise to a solution of tert-butyl 4-(5-(methoxy(methyl)carbamoyl)pyrazin-2-yl)piperazine-1-carboxylate (compound 31c, 300 mg, 854 μmol) in THF (5 mL) at −78° C. After the reaction mixture was stirred for 2 hours, the reaction was quenched with water and extracted with EA. The organic layer was dried and concentrated to give compound 31d as a light yellow oil (150 mg). LCMS(M+H)⁺: 293.

### Step 4: Preparation of tert-butyl 4-(5-((((3R,5S)-1-(8-cyanoquinolin-5-yl)-5-methylpiperidin-3-yl)amino)methyl)pyrazin-2-yl)piperazine-1-carboxylate (Compound 31e)

A mixture of 5-((3R,5S)-3-amino-5-methylpiperidin-1-yl)quinoline-8-carbonitrile (compound 1c, 100 mg, 375 μmol), tert-butyl 4-(5-formylpyrazin-2-yl)piperazine-1-carboxylate (165 mg, 563 μmol), TEA (52.3 μL, 375 μmol), AcOH (21.5 μL, 375 μmol and sodium triacetoxyborohydride (239 mg, 1.13 mmol) in DCM (15 mL) was stirred at rt for 2 hours. The reaction mixture was poured into 10 mL of aqueous sat. NaHCO₃ solution and extracted with EtOAc (20 mL). The organic layer was concentrated and the crude residue was purified by preparative HPLC to give compound 31e as a light yellow solid (80 mg). LCMS(M+H)⁺: 543.

### Step 5: Preparation of 5-[(3S,5R)-3-methyl-5-[(5-piperazin-1-ylpyrazin-2-yl)methylamino]-1-piperidyl]quinoline-8-carbonitrile (Example 31)

A mixture of tert-butyl 4-(5-((((3R,5S)-1-(8-cyanoquinolin-5-yl)-5-methylpiperidin-3-yl)amino)methyl)pyrazin-2-yl)piperazine-1-carboxylate (compound 31e, 80 mg, 147 μmol in 1 M HCl in EA (10 mL) was stirred at rt for 16 hours. The solution was concentrated to give Example 31 as an orange solid (70 mg). LCMS(M+H)⁺: 443, ¹H NMR (400 MHz, DMSO-d₆) δ ppm: 9.76 (br d, J=4.8 Hz, 2H), 9.51 (br s, 2H), 9.07 (dd, J=1.6, 4.2 Hz, 1H), 8.56-8.22 (m, 4H), 7.71 (dd, J=4.2, 8.6 Hz, 1H), 7.29 (d, J=8.2 Hz, 1H), 4.39-4.20 (m, 2H), 3.97 (br d, J=10.5 Hz, 1H), 3.93-3.84 (m, 4H), 3.62 (br s, 1H), 3.40 (br d, J=9.4 Hz, 1H), 3.19 (br s, 4H), 2.93 (t, J=11.2 Hz, 1H), 2.56 (s, 1H), 2.39 (br d, J=12.0 Hz, 1H), 2.01 (br d, J=13.7 Hz, 1H), 1.37 (br d, J=12.0 Hz, 1H), 0.99 (d, J=6.5 Hz, 3H).

### Example 32

The following tests were carried out in order to determine the activity of the compounds of this invention in HEK293-Blue-hTLR-7/8/9 cells assay.

HEK293-Blue-hTLR-7 Cells Assay:

A stable HEK293-Blue-hTLR-7 cell line was purchased from InvivoGen (Cat. #: hkb-htlr7, San Diego, Calif., USA). These cells were originally designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression was declined by TLR7 antagonist under the stimulation of a ligand, such as R848 (Resiquimod), for incubation of 20 hrs. The cell culture supernatant SEAP reporter activity was determined using QUANTI-Blue™ kit (Cat. #: rep-qb1, Invivogen, San Diego, Calif., USA) at a wavelength of 640 nm, a detection medium that turns purple or blue in the presence of alkaline phosphatase.

HEK293-Blue-hTLR7 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620-655 nm using a spectrophotometer. The signalling pathway that TLR7 activation leads to downstream NF-κB activation has been widely accepted, and therefore similar reporter assay was modified for evaluating TLR7 antagonist.

HEK293-Blue-hTLR-8 Cells Assay:

A stable HEK293-Blue-hTLR-8 cell line was purchased from InvivoGen (Cat. #: hkb-htlr8, San Diego, Calif., USA). These cells were originally designed for studying the stimulation of human TLR8 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR8 cells with TLR8 ligands. Therefore the reporter expression was declined by TLR8 antagonist under the stimulation of a ligand, such as R848, for incubation of 20 hrs. The cell culture supernatant SEAP reporter activity was determined using QUANTI-Blue™kit (Cat. #: rep-qb1, Invivogen, San Diego, Calif., USA) at a wavelength of 640 nm, a detection medium that turns purple or blue in the presence of alkaline phosphatase.

HEK293-Blue-hTLR8 cells were incubated at a density of 250,000450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 60 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signalling pathway that TLR8 activation leads to downstream NF-κB activation has been widely accepted, and therefore similar reporter assay was modified for evaluating TLR8 antagonist.

HEK293-Blue-hTLR-9 Cells Assay:

A stable HEK293-Blue-hTLR-9 cell line was purchased from InvivoGen (Cat. #: hkb-htlr9, San Diego, Calif., USA). These cells were originally designed for studying the stimulation of human TLR9 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR9 cells with TLR9 ligands. Therefore the reporter expression was declined by TLR9 antagonist under the stimulation of a ligand, such as ODN2006 (Cat. #: tlr1-2006-1, Invivogen, San Diego, Calif., USA), for incubation of 20 hrs. The cell culture supernatant SEAP reporter activity was determined using QUANTI-Blue™ kit (Cat. #: rep-qb1, Invivogen, San Diego, Calif., USA) at a wavelength of 640 nm, a detection medium that turns purple or blue in the presence of alkaline phosphatase.

HEK293-Blue-hTLR9 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM ODN2006 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signalling pathway that TLR9 activation leads to downstream NF-κB activation has been widely accepted, and therefore similar reporter assay was modified for evaluating TLR9 antagonist.

The compounds of formula (I) have human TLR7 and/or TLR8 inhibitory activities (IC₅₀ value) <1 μM, particularly <0.1 μM. Moreover, some compounds also have human TLR9 inhibitory activity <1 μM, particularly <0.2 μM. Activity data of the compounds of the present invention were shown in Table 2.

TABLE 2
The activity of the compounds of present invention
in HEK293-Blue-hTLR-7/8/9 cells assays

| | TLR7 IC₅₀ | TLR8 IC₅₀ | TLR9 IC₅₀ |
|---|---|---|---|
| Example | (μM) | (μM) | (μM) |
| R1 | 0.089 | 0.020 | 1.872 |
| (reference | | | |
| compound) | | | |
| R2 | 0.048 | 0.11 | 8.658 |
| (reference | | | |
| compound) | | | |
| 1 | 0.016 | <0.0032* | 0.055 |
| 2 | 0.032 | 0.006 | 0.075 |
| 3 | 0.030 | 0.004 | 0.077 |
| 4 | 0.013 | <0.0032 | 0.094 |
| 5 | 0.017 | 0.006 | 0.068 |
| 6 | 0.042 | 0.004 | 0.070 |
| 7 | 0.016 | <0.0032 | 0.069 |
| 8 | 0.039 | 0.009 | 0.088 |
| 9 | 0.015 | 0.005 | 0.095 |
| 10 | 0.007 | <0.0032 | 0.068 |
| 11 | 0.015 | <0.0032 | 0.082 |
| 12 | 0.007 | <0.0032 | 0.073 |
| 13 | 0.019 | 0.006 | 0.163 |
| 15 | 0.084 | 0.010 | 0.126 |
| 16 | 0.011 | <0.0032 | 0.077 |
| 17 | 0.007 | <0.0032 | 0.060 |
| 18 | 0.011 | <0.0032 | 0.116 |
| 21 | 0.063 | 0.042 | 0.094 |
| 22 | 0.023 | 0.005 | 0.183 |
| 23 | 0.023 | 0.004 | 0.092 |
| 24 | 0.052 | <0.0032 | 0.070 |
| 25 | 0.015 | <0.0032 | 0.061 |
| 26 | 0.091 | 0.003 | 0.055 |
| 28 | 0.012 | <0.0032 | 0.048 |
| 29 | 0.008 | <0.0032 | 0.032 |
| 30 | 0.016 | <0.0032 | 0.033 |
| 31 | 0.003 | <0.0032 | 0.047 |

*0.0032 is the lowest tested concentration for TLR7 and TLR8

### Example 33

### Human Microsome Stability Assay

The human microsomal stability assay is used for early assessment of metabolic stability of a test compound in human liver microsomes. Compounds with high metabolic stability are considered to be desirable as they can provide favorable in vivo PK profiles and thus sufficient exposure in the targeted tissues or organs

Human liver microsomes (Cat. NO.: 452117, Corning, USA; Cat. NO.: H2610, Xenotech, USA) were preincubated with test compound for 10 minutes at 37° C. in 100 mM potassium phosphate buffer, pH 7.4. The reactions were initiated by adding NADPH regenerating system. The final incubation mixtures contained 1 μM test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation times of 0, 3, 6, 9, 15 and 30 minutes at 37° C., 300 μL of cold acetonitrile (including internal standard) was added to 100 μL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. The compounds of present invention showed good human liver microsome stability determined in the above assay.

TABLE 3
Human liver microsome stability of
the compounds of present invention

| Example No. | HLM (mL/min/Kg) |
|---|---|
| R1 | 17.3 |
| (reference compound) | |
| R2 | 18.4 |
| (reference compound) | |
| 1 | 7.3 |
| 2 | 7.0 |
| 10 | 7.4 |
| 12 | 6.2 |
| 17 | 6.8 |
| 21 | 6.2 |
| 23 | 7.3 |

1. A compound of formula (I), 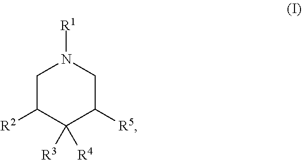
wherein
R¹ is 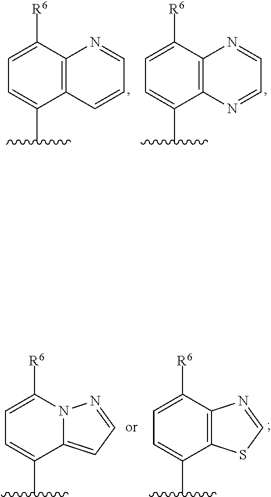
wherein R⁶ is cyano or halogen;
R² is C₁₋₆alkyl;
R³ is H or halogen;
R⁴ is H, halogen or hydroxy;
R⁵ is 1,3-dihydropyrrolo[3,4-c]pyridinyl substituted by piperazinyl;
2,3,3a,7a-tetrahydro-1H-indenylamino substituted by piperazinyl;
3,4-dihydro-1H-isoquinolinyl substituted by piperazinyl;
5,7-dihydropyrrolo[3,4-b]pyridinyl substituted by piperazinyl;
5,7-dihydropyrrolo[3,4-d]pyrimidinyl substituted by piperazinyl;
isoindolinyl substituted by piperazinyl; or
—NR^{5a}R^{5b}; wherein
R^{5a} is H or C₁₋₆alkyl;
R^{5b} is 1,2,3,4-tetrahydroisoquinolinylC₁₋₆ alkyl;
5,6,7,8-tetrahydro-1,6-naphthyridinylC₁₋₆alkyl;
isoindolinylC₁₋₆alkyl;
phenyl(hydroxy)C₁₋₆ alkyl substituted by piperazinyl;
phenylC₁₋₆alkyl, said phenylC₁₋₆alkyl being substituted by one or two substituents independently selected from (C₁₋₆ alkyl)₂pyridinyl, aminopiperidinyl, aminopyrrolidinyl, C₁₋₆alkylpiperazinyl, halogen, piperazinyl and piperidinyl;
pyrazinylC₁₋₆ alkyl substituted by piperazinyl;
pyridazinylC₁₋₆ alkyl substituted by piperazinyl;
pyridinylC₁₋₆ alkyl substituted by piperazinyl or C₁₋₆alkylpiperazinyl; or
pyrimidinylC₁₋₆ alkyl substituted by piperazinyl or C₁₋₆alkylpiperazinyl;
or a pharmaceutically acceptable salt, enantiomer or diastereomer thereof.

2. A compound of formula (Ia), 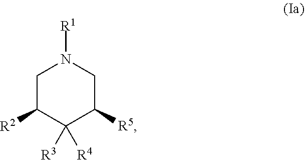
wherein
R¹ is 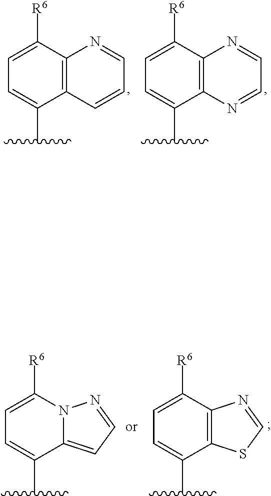
wherein R⁶ is cyano or halogen;
R² is C₁₋₆alkyl;
R³ is H or halogen;
R⁴ is H, halogen or hydroxy;
R⁵ is 1,3-dihydropyrrolo[3,4-c]pyridinyl substituted by piperazinyl;
2,3,3a,7a-tetrahydro-1H-indenylamino substituted by piperazinyl;
3,4-dihydro-1H-isoquinolinyl substituted by piperazinyl;
5,7-dihydropyrrolo[3,4-b]pyridinyl substituted by piperazinyl;
5,7-dihydropyrrolo[3,4-d]pyrimidinyl substituted by piperazinyl;
isoindolinyl substituted by piperazinyl; or
—NR^{5a}R^{5b}; wherein
R^{5a} is H or C₁₋₆alkyl;
R^{5b} is 1,2,3,4-tetrahydroisoquinolinylC₁₋₆ alkyl;
5,6,7,8-tetrahydro-1,6-naphthyridinylC₁₋₆alkyl;
isoindolinylC₁₋₆ alkyl;
phenyl(hydroxy)C₁₋₆ alkyl substituted by piperazinyl;
phenylC₁₋₆alkyl, said phenylC₁₋₆alkyl being substituted by one or two substituents independently selected from (C₁₋₆ alkyl)₂pyridinyl, aminopiperidinyl, aminopyrrolidinyl, C₁₋₆alkylpiperazinyl, halogen, piperazinyl and piperidinyl;
pyrazinylC₁₋₆ alkyl substituted by piperazinyl;
pyridazinylC₁₋₆ alkyl substituted by piperazinyl;
pyridinylC₁₋₆ alkyl substituted by piperazinyl or C₁₋₆alkylpiperazinyl; or
pyrimidinylC₁₋₆ alkyl substituted by piperazinyl or C₁₋₆alkylpiperazinyl;
or a pharmaceutically acceptable salt, enantiomer or diastereomer thereof.

3. A compound according to claim 1 or 2, wherein
R¹ is 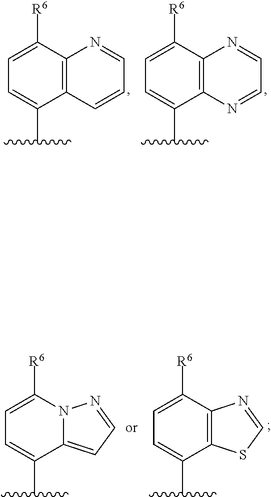
wherein R⁶ is cyano or halogen;
R² is C₁₋₆alkyl;
R³ is H;
R⁴ is H;
R⁵ is 3,4-dihydro-1H-isoquinolinyl substituted by piperazinyl;
5,7-dihydropyrrolo[3,4-b]pyridinyl substituted by piperazinyl;
isoindolinyl substituted by piperazinyl; or
—NR^{5a}R^{5b}; wherein
R^{5a} is H or C₁₋₆alkyl;
R^{5b} is 1,2,3,4-tetrahydroisoquinolinylC₁₋₆ alkyl;
5,6,7,8-tetrahydro-1,6-naphthyridinylC₁₋₆alkyl;
phenylC₁₋₆alkyl, said phenylC₁₋₆alkyl being substituted by one or two substituents independently selected from (C₁₋₆ alkyl)₂pyridinyl, aminopiperidinyl, aminopyrrolidinyl, C₁₋₆alkylpiperazinyl, halogen, piperazinyl and piperidinyl;
pyrazinylC₁₋₆ alkyl substituted by piperazinyl;
pyridinylC₁₋₆ alkyl substituted by piperazinyl or C₁₋₆alkylpiperazinyl; or
pyrimidinylC₁₋₆ alkyl substituted by piperazinyl or C₁₋₆alkylpiperazinyl;
or a pharmaceutically acceptable salt, enantiomer or diastereomer thereof.

4. A compound according to claim 3, wherein
R¹ is 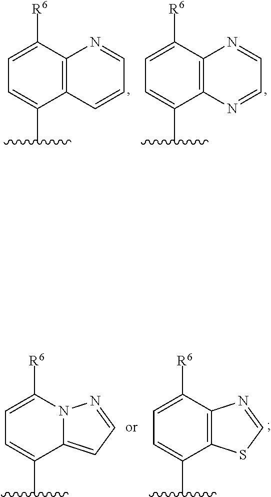
wherein R⁶ is cyano or chloro;
R² is methyl;
R³ is H;
R⁴ is H;
R⁵ is ((aminopiperidinyl)phenyl)methylamino; ((aminopyrrolidinyl)phenyl)methylamino; ((dimethylpyridinyl)phenyl)methylamino; ((methylpiperazinyl)phenyl)methylamino; ((methylpiperazinyl)pyridinyl)methylamino; ((methylpiperazinyl)pyrimidinyl)methylamino; (5,6,7,8-tetrahydro-1,6-naphthyridinyl)methylamino; (fluoro(piperazinyl)phenyl)methylamino; (piperazinylphenyl)ethylamino; (piperazinylphenyl)methylamino; (piperazinylpyrazinyl)methylamino; (piperazinylpyridinyl)methylamino; (piperazinylpyrimidinyl)methylamino; (piperidinylphenyl)methylamino; 1,2,3,4-tetrahydroisoquinolinylmethylamino; methyl((piperazinylphenyl)methyl)amino; piperazinyl-3,4-dihydro-1H-isoquinolinyl; piperazinyl-5,7-dihydropyrrolo[3,4-b]pyridinyl; or piperazinylisoindolinyl;
or a pharmaceutically acceptable salt, enantiomer or diastereomer thereof.

5. A compound according to claim 3, wherein R¹ is 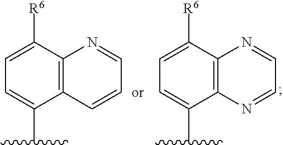
wherein R⁶ is cyano.

6. A compound according to claim 5, wherein R² is methyl.

7. A compound according to claim 6, wherein
R⁵ is 5,7-dihydropyrrolo[3,4-b]pyridinyl substituted by piperazinyl; or
—NR^{5a}R^{5b}; wherein
R^{5a} is H;
R^{5b} is phenylC₁₋₆ alkyl substituted by piperazinyl; or pyrimidinylC₁₋₆ alkyl substituted by piperazinyl.

8. A compound according to claim 7, wherein R⁵ is piperazinyl-5,7-dihydropyrrolo[3,4-b]pyridinyl; (piperazinylphenyl)methylamino or (piperazinylpyrimidinyl)methylamino.

9. A compound according to claim 1 or 2, wherein
R¹ is 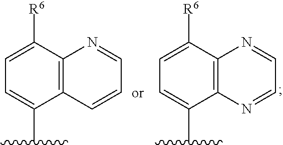
wherein R⁶ is cyano;
R² is C₁₋₆alkyl;
R³ is H;
R⁴ is H;
R⁵ is R⁵ is 5,7-dihydropyrrolo[3,4-b]pyridinyl substituted by piperazinyl; or
—NR^{5a}R^{5b}; wherein
R^{5a} is H;
R^{5b} is phenylC₁₋₆ alkyl substituted by piperazinyl; or pyrimidinylC₁₋₆ alkyl substituted by piperazinyl;
or a pharmaceutically acceptable salt, enantiomer or diastereomer thereof.

10. A compound according to claim 9, wherein
R¹ is 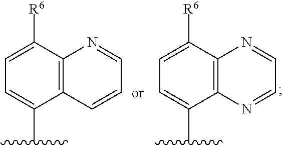
wherein R⁶ is cyano;
R² is methyl;
R³ is H;
R⁴ is H;
R⁵ is piperazinyl-5,7-dihydropyrrolo[3,4-b]pyridinyl; (piperazinylphenyl)methylamino or (piperazinylpyrimidinyl)methylamino;
or a pharmaceutically acceptable salt, enantiomer or diastereomer thereof.

11. A compound according to claim 1 or 2, selected from:
5-[(3S,5R)-3-methyl-5-[(4-piperazin-1-ylphenyl)methylamino]-1-piperidyl]quinoline-8-carbonitrile;
5-[(3S,5R)-3-methyl-5-[[4-(4-piperidyl)phenyl]methylamino]-1-piperidyl]quinoline-8-carbonitrile;
8-[(3S,5R)-3-methyl-5-[(4-piperazin-1-ylphenyl)methylamino]-1-piperidyl]quinoxaline-5-carbonitrile;
5-[(3R,5S)-3-[(2-fluoro-4-piperazin-1-yl-phenyl)methylamino]-5-methyl-1-piperidyl]quinoline-8-carbonitrile;
5-[(3S,5R)-3-methyl-5-[(5-piperazin-1-yl-2-pyridyl)methylamino]-1-piperidyl]quinoline-8-carbonitrile;
5-[(3S,5R)-3-methyl-5-[[3-(4-methylpiperazin-1-yl)phenyl]methylamino]-1-piperidyl]quinoline-8-carbonitrile;
5-[(3S,5R)-3-methyl-5-[[4-(4-methylpiperazin-1-yl)phenyl]methylamino]-1-piperidyl]quinoline-8-carbonitrile;
5-[(3S,5R)-3-methyl-5-[(3-piperazin-1-ylphenyl)methylamino]-1-piperidyl]quinoline-8-carbonitrile;
5-[(3S,5R)-3-methyl-5-[methyl-[(4-piperazin-1-ylphenyl)methyl]amino]-1-piperidyl]quinoline-8-carbonitrile;
5-[(3S,5R)-3-methyl-5-[methyl-[(4-piperazin-1-ylphenyl)methyl]amino]-1-piperidyl]quinoline-8-carbonitrile;
5-[(3S,5R)-3-methyl-5-(1,2,3,4-tetrahydroisoquinolin-6-ylmethylamino)-1-piperidyl]quinoline-8-carbonitrile;
5-[(3S,5R)-3-methyl-5-(5-piperazin-1-ylisoindolin-2-yl)-1-piperidyl]quinoline-8-carbonitrile;
5-[(3R,5S)-3-[[4-(3-aminopyrrolidin-1-yl)phenyl]methylamino]-5-methyl-1-piperidyl]quinoline-8-carbonitrile;
5-[(3R,5S)-3-[[4-(3-amino-1-piperidyl)phenyl]methylamino]-5-methyl-1-piperidyl]quinoline-8-carbonitrile;
5-[(3R,5S)-3-[[4-(2,6-dimethyl-4-pyridyl)phenyl]methylamino]-5-methyl-1-piperidyl]quinoline-8-carbonitrile;
5-[(3S,5R)-3-methyl-5-[[2-(4-methylpiperazin-1-yl)pyrimidin-5-yl]methylamino]-1-piperidyl]quinoline-8-carbonitrile;
5-[(3S,5R)-3-methyl-5-[(2-piperazin-1-ylpyrimidin-5-yl)methyl amino]-1-piperidyl]quinoline-8-carbonitrile;
7-[(3S,5R)-3-methyl-5-[[5-(4-methylpiperazin-1-yl)-2-pyridyl]methylamino]-1-piperidyl]-1,3-benzothiazole-4-carbonitrile;
4-[(3S,5R)-3-methyl-5-[(4-piperazin-1-ylphenyl)methylamino]-1-piperidyl]pyrazolo[1,5-a]pyridine-7-carbonitrile;
(3R,5S)-1-(8-chloro-5-quinolyl)-5-methyl-N-[(4-piperazin-1-ylphenyl)methyl]piperidin-3-amine;
5-[(3S,5R)-3-methyl-5-(3-piperazin-1-yl-5,7-dihydropyrrolo[3,4-b]pyridin-6-yl)-1-piperidyl]quinoline-8-carbonitrile;
5-[cis-3-methyl-5-(6-piperazin-1-yl-3,4-dihydro-1H-isoquinolin-2-yl)-1-piperidyl]quinoline-8-carbonitrile;
5-[(3S,5R)-3-methyl-5-[(5-piperazin-1-ylpyrimidin-2-yl)methyl amino]-1-piperidyl]quinoline-8-carbonitrile;
5-[(3S,5R)-3-methyl-5-[1-(4-piperazin-1-ylphenyl)ethylamino]-1-piperidyl]quinoline-8-carbonitrile;
5-[(3S,5R)-3-methyl-5-[[2-[(3R)-3-methylpiperazin-1-yl]pyrimidin-5-yl]methyl amino]-1-piperidyl]quinoline-8-carbonitrile;
5-[(3S,5R)-3-methyl-5-[[2-[(3S)-3-methylpiperazin-1-yl]pyrimidin-5-yl]methylamino]-1-piperidyl]quinoline-8-carbonitrile;
5-[(3S,5R)-3-methyl-5-(5,6,7,8-tetrahydro-1,6-naphthyridin-2-ylmethylamino)-1-piperidyl]quinoline-8-carbonitrile; and
5-[(3S,5R)-3-methyl-5-[(5-piperazin-1-ylpyrazin-2-yl)methylamino]-1-piperidyl]quinoline-8-carbonitrile;
or a pharmaceutically acceptable salt, enantiomer or diastereomer thereof.

12. A process for the preparation of a compound according to any one of claims 1 to 11 comprising any of the following steps:
a) the nucleophilic substitution of compound of formula (V), 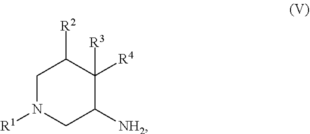
with compound (VI) in the presence of a base;
b) the reductive amination of compound of formula (V), 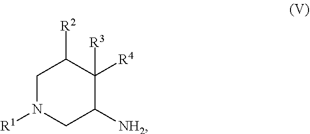
with compound (VII);
c) the Buchwald-Hartwig amination of compound of formula (X), 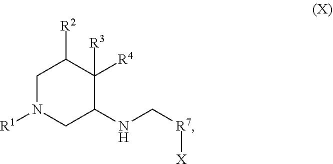
with an amine;
d) the Suzuki-Miyaura reaction of compound of formula (X), 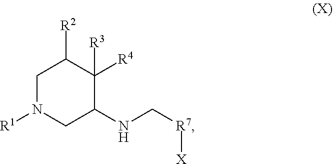
with an boronic acid;
e) the Buchwald-Hartwig amination of compound of formula (XIII), 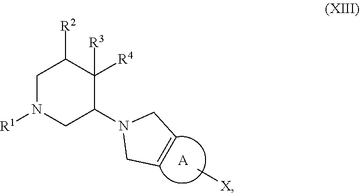
with an amine;
f) the Buchwald-Hartwig amination of compound of formula (XIX), 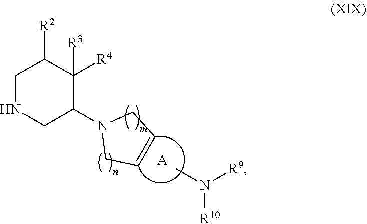
with halide (II);
wherein the base in step a) is K₂CO₃ or DIPEA, R⁷ is heterocyclyl; R⁸ is C₁₋₆ alkyl or hydroxyC₁₋₆ alkyl; A is heterocyclyl; m is 1 or 2; n is 1 or 2; R⁹ and R¹⁰ together with the carbon atoms they are attached to form a heterocyclyl; R¹ to R⁴ are defined as in any one of claims 1 to 10.

13. A compound or pharmaceutically acceptable salt, enantiomer or diastereomer according to any one of claims 1 to 11 for use as therapeutically active substance.

14. A pharmaceutical composition comprising a compound in accordance with any one of claims 1 to 11 and a therapeutically inert carrier.

15. The use of a compound according to any one of claims 1 to 11 for the treatment or prophylaxis of systemic lupus erythematosus or lupus nephritis.

16. The use of a compound according to any one of claims 1 to 11 for the preparation of a medicament for the treatment or prophylaxis of systemic lupus erythematosus or lupus nephritis.

17. The use of a compound according to any one of claims 1 to 11 as the TLR7 or TLR8 or TLR9 antagonist.

18. The use of a compound according to any one of claims 1 to 11 as the TLR7 and TLR8 antagonist.

19. A compound or pharmaceutically acceptable salt, enantiomer or diastereomer according to any one of claims 1 to 11 for the treatment or prophylaxis of systemic lupus erythematosus or lupus nephritis.

20. A compound or pharmaceutically acceptable salt, enantiomer or diastereomer according to any one of claims 1 to 11, when manufactured according to a process of claim 12.

21. A method for the treatment or prophylaxis of systemic lupus erythematosus or lupus nephritis, which method comprises administering a therapeutically effective amount of a compound as defined in any one of claims 1 to 11.
